(19) United States
(12) Patent Application Publication
(10) Pub. No.: US 2017/0350856 A1
(43) Pub. Date: Dec. 7, 2017

(54) FIELD EFFECT TRANSISTOR AND SENSOR USING SAME

(71) Applicants: NIPPON KAYAKU KABUSHIKI KAISHA, Tokyo (JP); OSAKA UNIVERSITY, Osaka (JP)

(72) Inventors: Yoshihiro KOBAYASHI, Osaka (JP); Ryota NEGISHI, Osaka (JP); Hiroto KASE, Osaka (JP); Michiharu ARIFUKU, Tokyo (JP); Noriko KIYOYANAGI, Tokyo (JP); Tomio MORINO, Tokyo (JP)

(73) Assignees: NIPPON KAYAKU KABUSHIKI KAISHA, Tokyo (JP); OSAKA UNIVERSITY, Osaka (JP)

(21) Appl. No.: 15/501,991

(22) PCT Filed: Aug. 6, 2015
(86) PCT No.: PCT/JP2015/072398

§ 371 Date: Jul. 5, 2017

(30) Foreign Application Priority Data
Aug. 8, 2014 (JP) 2014-162737

(51) Int. Cl. G01N 27/414; C01B 32/16; C01B 32/168; H01L 51/05; G01N 33/543; C12Q 1/68; B82Y 35/00; B82Y 15/00

(52) U.S. Cl. CPC G01N 27/4148; G01N 33/54346; C01B 32/16; C12Q 1/6825; G01N 27/4145; C01B 32/168; G01N 33/5438; H01L 51/0558; C01B 2202/02; B82Y 15/00; B82Y 35/00; Y10S 977/746; Y10S 977/75; Y10S 977/843; Y10S 977/847; Y10S 977/92

(57) ABSTRACT

A field effect transistor and a sensor using the field effect transistor is provided. The field effect transistor can be manufactured so as to have uniform properties by simple steps at low costs, and can stably detect, when used as a sensor, a very small amount of analyte with a high sensitivity while the properties are hardly deteriorated. A channel of the field effect transistor is constituted by a single-walled carbon nanotube thin film that is grown, by a chemical vapor deposition method, using particles of a nonmetallic material as growth nuclei, the nonmetallic material containing 500 mass ppm or less metallic impurities that contain a metal and its compounds.

### TECHNICAL FIELD

The present invention relates to a field effect transistor and a sensor using the field effect transistor.

### BACKGROUND ART

Recently, as high performance analyses and medical diagnoses are more simplified and highly developed, sensors such as chemical sensors and biosensors become more essential. The chemical sensors are to detect ions in solutions, and the biosensors are capable of detecting proteins, such as antigens and antibodies, and DNA (deoxyribonucleic acid), which are deeply involved in life phenomena.

Examples of the conventionally proposed chemical sensors and biosensors include a sensor in which a thin film having a reaction group to selectively react with a specific molecule is formed on an electrode so as to measure a change in a potential when the specific molecules adsorb onto the thin film. More specifically, a biosensor is proposed, in which a thin film having glucose oxidase is formed on the electrode so as to detect the amount of glucose by measuring a change in a current value accompanying the oxidation reaction with the glucose (see Patent Document 1, and Non-Patent Documents 1 and 2).

However, since these chemical sensors and biosensors are to directly detect the current value accompanying the chemical reaction, the detection sensitivity is reduced, which leads to the difficulty in detection of specific molecules at low concentration (for example, glucose). Thus, it is difficult for the chemical sensors and the biosensors to sufficiently exert their feature of high selectivity.

As another sensor recently proposed, there is a sensor for performing optical detection using surface plasmon resonance. However, this type of sensor requires an accurate and complicated system, which results in increase in cost. Also, there is a problem that noise is increased when nonspecific adsorption molecules exist.

A sensor using a field effect transistor is considered in order to resolve the above problems. Examples of the sensors using the field effect transistor include: a chemical sensor having a sensing part as a configuration constituted by one dimensional carbon structures of carbon nanotubes and the like that are vertically aligned (see Patent Document 2); a sensor having a structure in which a carbon nanotube is loosely disposed as a channel (see Patent Document 3); a sensor having a specific structure in which a channel is made of ultrafine fibers such as carbon nanotubes and in which a space is disposed between the channel and a substrate (see Patent Document 4); and a sensor having a channel made of carbon nanotubes aligned between the electrodes (see Non-Patent Document 3).

### CITATION LIST

### Patent Literature

[Patent Literature 1] JP H10-260156 A
[Patent Literature 2] JP 2004-085392 A
[Patent Literature 3] JP 4669213
[Patent Literature 4] JP 4774476

### Non-Patent Literature

[Non-Patent Literature 1] Yabuki, Shinohara, and Aizawa, Journal of the Chemical Society, Chemical Communications, 1989, pp. 945-946.
[Non-Patent Literature 2] Alexander Star, Jean-Christophe P. Gabriel, Keith Bradley, and George Gruner, Nano Letters, 2003, volume 3, no. 4, pp. 459-463.
[Non-Patent Document 3] J. Phys. Chem., C 2012, 116, pp. 19490-19495.

### SUMMARY OF INVENTION

### Technical Problem

In the sensors using the conventional field effect transistor that are proposed in Patent Documents 2 to 4 and Non-Patent Document 3, when making the transistor, the step of, for example, forming the channel is complicated and sophisticated. Furthermore, it is difficult to obtain a sufficient detection sensitivity. Especially, in a sensor for using as a biosensor (simply referred to as “biosensor”), a target analyte should be selectively detected. Therefore, the sensor itself should be chemically stable while it should have properties as a highly sensitive sensor that is capable of stably detecting the very small amount of analyte.

However, when using, as a transducer of a biosensor, a conventional field effect transistor in which the carbon nanotubes including a catalyst metal for growth are used, the catalyst metal for growth of the carbon nanotubes reacts with oxygen in the atmosphere, which causes changes with time. Thus, the properties of the sensor may be deteriorated. Also, the carbon nanotubes perform the oxidation reaction or the reduction reaction in the solution, which leads to decrease or destabilization in sensing sensitivity.

Also, in the conventional art in which single-walled carbon nanotubes are used for the channel structure of the field effect transistor, variation in properties of the carbon nanotubes widely affects the sensing sensitivity. Accordingly, it is difficult to manufacture the sensors having uniform properties.

In consideration of the above circumstances, an object of the present invention is to provide: a field effect transistor that can be manufactured so as to have uniform properties by simple steps at low costs, and that can stably detect, when used as a sensor, the very small amount of analyte with a high sensitivity while the properties are hardly deteriorated; and a sensor using the field effect transistor.

### Solution to Problem

In order to resolve the above problems, a field effect transistor of the present invention includes: a source electrode; a drain electrode; a channel formed between the source electrode and the drain electrode; and a gate electrode. A channel is constituted by a single-walled carbon nanotube thin film that is grown, by a chemical vapor deposition method, using particles of a nonmetallic material as growth nuclei, the nonmetallic material containing 500 mass ppm or less metallic impurities that contain a metal and its compounds.

In the above-described field effect transistor of the present invention, the channel is constituted by the single-walled carbon nanotube thin film that is grown, by the chemical vapor deposition method, using the particles of a nonmetallic material as the growth nuclei, the nonmetallic material containing 500 mass ppm or less metallic impurities that contain a metal and its compounds. Thus, a carrier mobility is improved and good transistor properties can be obtained. Also, it is possible to manufacture the field effect transistors having uniform properties by simple steps at low costs.

### Advantageous Effects of Invention

In the present invention, a channel is constituted by the single-walled carbon nanotube thin film that is grown, by the chemical vapor deposition method, using particles of a nonmetallic material as the growth nuclei, the nonmetallic material containing 500 mass ppm or less metallic impurities that contain a metal and its compounds. In this way, it is possible to provide: a field effect transistor that can be manufactured so as to have uniform properties by simple steps at low costs, and can stably detect, when used as a sensor, a very small amount of analyte with a high sensitivity while the properties are hardly deteriorated; and a sensor using the field effect transistor.

### BRIEF DESCRIPTION OF DRAWINGS

FIG. 1A is a schematic cross-sectional view showing a configuration of a field effect transistor according to a first embodiment. FIG. 1B is a schematic plan view showing the configuration of the field effect transistor according to the first embodiment.

FIG. 2 is a pattern diagram showing a CVD reactor for growing a single-walled carbon nanotube thin film.

FIG. 3 is a pattern diagram showing a CVD reactor with multiple temperature zones for growing a single-walled carbon nanotube thin film.

FIG. 4A is a graph showing a Raman spectrum of the single-walled carbon nanotube thin film in Example 1. FIG. 4B is a graph showing a Raman spectrum of the single-walled carbon nanotube thin film in Example 2.

FIG. 5A is a graph showing a Raman spectrum of the single-walled carbon nanotube thin film in Comparative Example 1. FIG. 5B is a graph showing a Raman spectrum of the single-walled carbon nanotube thin film in Comparative Example 2.

FIG. 6 is a pattern diagram showing a configuration of a sample of the field effect transistor for evaluating a carrier mobility μ.

FIG. 7 is a graph made by plotting each carrier mobility of the corresponding field effect transistor in Example 1 and Comparative Example 2.

FIG. 8 is a graph showing changes in the carrier mobility and the ratio I(G)/I(Si) of a single-walled carbon nanotube thin film field effect transistor according to the thickness of hydrogenated nanodiamond and to the growth condition time of the single-walled carbon nanotubes.

FIG. 9 is a schematic diagram showing a configuration of a sensor according to a second embodiment.

FIG. 10 is a graph showing pH measurement results by the sensor.

FIG. 11 is a schematic diagram showing a configuration of a biosensor according to a third embodiment.

FIG. 12 is a pattern diagram showing formation of an electric double layer in the biosensor.

FIG. 13A is a pattern diagram showing a potential before an analyte 33 adsorbs onto a surface of a single-walled carbon nanotube thin film 12 of the biosensor. FIG. 13B is a pattern diagram showing a potential after the analyte 33 adsorbs onto the surface of the single-walled carbon nanotube thin film 12 of the biosensor.

FIG. 14A is a graph showing a change in a reference potential of the gate voltage and a change in a source-drain current, which indicates the change in the relation between the gate voltage and the source-drain current according to the change in the reference potential of the gate voltage. FIG. 14B is a graph showing a change in the reference potential of the gate voltage and a change in the source-drain current, which indicates the change in the source-drain current before and after the adsorption.

FIG. 15 is a pattern diagram showing a BSA detection method by the biosensor.

FIG. 16 is a graph showing results of the BSA detection by the biosensor.

FIG. 17 is a graph showing results of IgE detection by the biosensor.

FIG. 18 is a graph showing results of the IgE detection in a low concentration region by the biosensor.

FIG. 19 is a graph plotting each carrier mobility of long single-walled carbon nanotubes and short single-walled carbon nanotubes.

FIG. 20A is a pattern diagram showing a configuration of a single-walled carbon nanotube thin film according to a fourth embodiment, which indicates the mixture of the single-walled carbon nanotubes having metallic properties. FIG. 20B is a pattern diagram showing the configuration of a single-walled carbon nanotube thin film according to the fourth embodiment, which indicates the case in which slits are formed so as to have strips.

FIG. 21 is a schematic diagram showing a configuration of a biosensor according to a fifth embodiment.

FIG. 22 is a schematic diagram showing a configuration of a biosensor according to a sixth embodiment.

FIG. 23 is a schematic diagram showing a configuration of a biosensor according to a seventh embodiment.

FIG. 24 is a schematic diagram showing a configuration of a biosensor according to an eighth embodiment.

FIG. 25 is a schematic diagram showing a configuration of a biosensor according to a ninth embodiment.

### DESCRIPTION OF EMBODIMENTS

Hereinafter, embodiments of the present invention will be described with reference to the drawings.

### First Embodiment

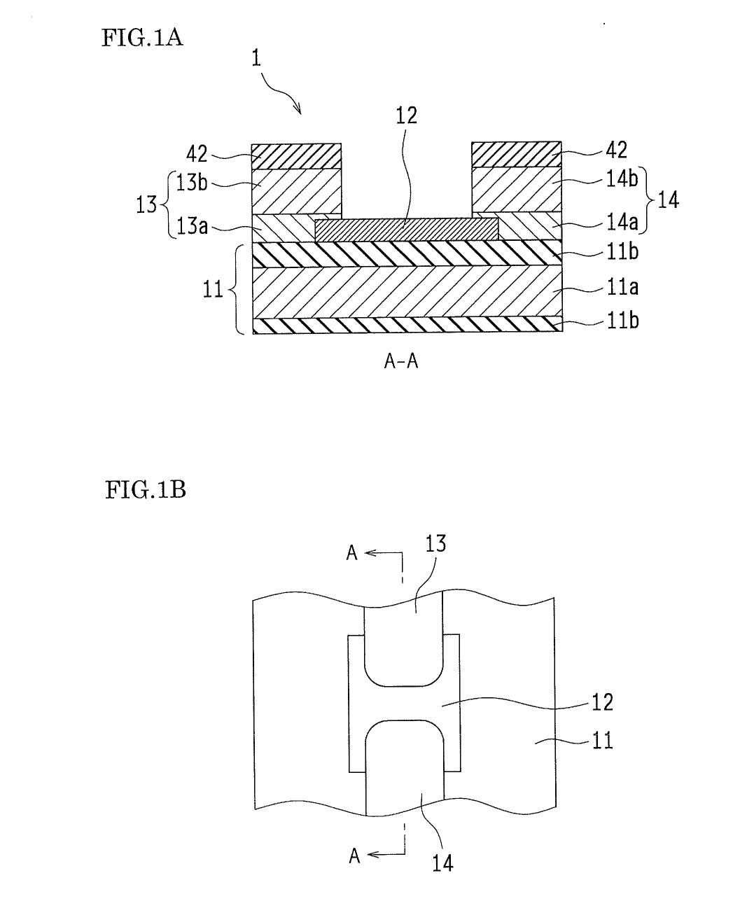

FIGS. 1A and 1B are schematic diagrams each showing a configuration of a field effect transistor according to the first embodiment of the present invention. FIG. 1A is a schematic cross-sectional view thereof, and FIG. 1B is a schematic plan view thereof. Also, FIG. 1A is the cross-sectional view taken from line A-A of FIG. 1B. As shown in FIGS. 1A and 1B, a field effect transistor 1 includes: a substrate 11; a substantially rectangular-shaped single-walled carbon nanotube thin film 12 that is formed on the substrate 11; a source electrode 13 and a drain electrode 14 that are formed on the substrate 11 so as to partially cover both end portions of the single-walled carbon nanotube thin film 12; and a gate electrode that is disposed outside (not shown).

The source electrode 13 and the drain electrode 14 are disposed so as to be separated from each other at a substantially center portion of the single-walled carbon nanotube thin film 12. The single-walled carbon nanotube thin film 12 in the above separation region constitutes a channel of the field effect transistor 1. Therefore, the separation between the source electrode 13 and the drain electrode 14 is the channel length of the field effect transistor 1, which is set, for example, to 5 μm. Also, the width of the source electrode 13 and the drain electrode 14 is the channel width of the field effect transistor 1, which is set, for example, to 10 μm.

At least parts of the substrate 11 that make contact with the source electrode 13 and the drain electrode 14 have electrically insulating property. Thus, the substrate 11 is, for example, a thermally oxidized silicon substrate in which a silicon oxide layer 11b is each formed on the surface and the rear surface of the silicon substrate 11a by oxidation. Since the material for substrate 11 is not particularly limited, any appropriate material can be used. Specifically, the insulating substrate that can be used for the substrate 11 may be composed of an inorganic compound such as silicon oxide, silicon nitride, aluminum oxide, titanium oxide or calcium fluoride, or an organic compound such as acrylic resin, polyimide or polytetrafluoroethylene (Teflon (registered trade mark)). Also, in place of the silicon substrate 11a, it is possible to use a single semiconductor substrate such as gallium arsenide, gallium nitride, zinc oxide, indium phosphide or silicon carbide, or to use any mixture thereof. By forming an insulating film on the surface of the above semiconductor substrate, it can be used as the substrate 11. Generally, in order to ensure the insulation, the thickness of the insulating film or the silicon oxide layer 11b that is formed on the surface of the semiconductor substrate is at least 10 nm or more.

As described later, when the single-walled carbon nanotube thin film 12 is directly synthesized on the substrate 11, it is preferable to use the substrate composed of the inorganic compound as the substrate 11 for its excellent heat resistance. When the substrate composed of the compound is used as the substrate 11, the single-walled carbon nanotube thin film 12 is synthesized on the substrate of the inorganic compound and then it is transferred onto the substrate of the organic compound.

The substrate 11 may have any shape. Generally, it has the shape of a flat plate, however, it may have the shape of a curved plate. Also, a flexible substrate made of a film and the like may be used as the substrate 11. The thickness of the substrate 11 is not particularly limited, however, it is preferable that the substrate 11 has, normally, the thickness of 10 μm or more, and more preferably has the thickness of 50 μm or more, in order to maintain the mechanical strength.

As described later, the single-walled carbon nanotube thin film 12 is made as a thin film of a single-walled carbon nanotube (CNT) aggregate, which is directly grown on the substrate 11 by the chemical vapor deposition method (CVD method), using particles of a nonmetallic material as growth nuclei, the nonmetallic material containing 500 mass ppm or less metallic impurities that contain a metal and its compounds.

The single-walled carbon nanotube thin film 12 of the present invention is formed using the above-described manufacturing method, and it is not clear what property (such as the structure or the material) causes the essential function and effect of the present invention. In order to evaluate the single-walled carbon nanotube thin film 12, for example, the quality, the purity, and the density thereof can be considered.

The quality of the single-walled carbon nanotube thin film 12 can be exemplarily expressed, in the Raman spectrum of the CNT, by the intensity ratio I(G)/I(D) of G band (Raman shift of about 1590 cm⁻¹) derived from the graphene structure of the CNT to D band (Raman shift of about 1350 cm⁻¹) derived from point defects and crystal single defects in the CNT wall. When the amorphous carbon content is increased, the intensity ratio I(G)/I(D) is decreased. The higher the value of I(G)/I(D) becomes, the higher the quality becomes.

As to the purity of the single-walled carbon nanotube thin film 12, it is preferable to contain less amorphous carbon or metallic impurities. Especially, by setting the metallic impurity content, which is identified by the chemical or physical means, to 500 mass ppm or less, or more preferably to 300 mass ppm or less, the single-walled carbon nanotube thin film 12 is suitably used for the purpose of the present invention.

Furthermore, the efficiency when growing the single-walled carbon nanotube thin film 12 can be expressed by the growth degree of the CNT. It can be exemplarily expressed, in the Raman spectrum of the CNT growing on the silicon substrate, by the intensity ratio I(G)/I(Si) of G band derived from the graphene structure to the Si band derived from the silicon. It is possible to consider that the higher the value of I(G)/I(Si) becomes, the higher the efficiency (yield) becomes.

When defining the density of the single-walled carbon nanotube thin film 12, for example, by the ratio I(G)/I(Si), preferably it falls within the range of 0.01 to 4.0, more preferably within the range of 0.01 to 2.0. When the ratio I(G)/I(Si) is excessively high, cross-linking is carried out in the channel region of the single-walled carbon nanotubes having the metal properties, which prevents the operation as the transistor. On the other hand, when the ratio I(G)/I(Si) is excessively low, the channel region is not formed and accordingly carriers do not flow, or the single-walled carbon nanotube thin film 12 has a high resistance that leads to deterioration of the transistor properties. Thus, it is preferable to form the CNT having a low density to a certain extent and to form the channel region with the long CNT.

Furthermore, in order to enhance the physical strength of the thin film, an overcoat layer of the inorganic compound or the organic compound having a thickness of several nm may be formed on the single-walled carbon nanotube thin film 12.

The source electrode 13 and the drain electrode 14 are multi-layered electrodes made by laminating, for example, respectively a Ti layer 13a and an Au layer 13b, and a Ti layer 14a and an Au layer 14b. Part of the each surface of the source electrode 13 and the drain electrode 14 may be coated by a coating layer 42 constituted by the insulating thin film made of alumina layer or silicon oxide film. In FIG. 1B, the coating layer 42 is omitted. Apart from the Ti and Au, the metal such as gold, platinum, titanium or palladium may be used as the electrode material to form the single layer. Also, two or more thereof may be appropriately combined so as to form the multi-layer structure. The insulating thin film for coating the surface of the source electrode 13 and the drain electrode 14 is not limited to alumina. The other insulating materials such as hafnium oxide and silicon dioxide may be used.

The source electrode 13 and the drain electrode 14 can be formed by publically known methods such as a screen printing, an ink jet printing and a photolithography. The interval between the source electrode 13 and the drain electrode 14 can be freely changed depending on the required sensor size and the integration degree. When the electrodes are formed, for example, by the photolithography, the interval is normally in the range of 1 μm to 1 mm, preferably in the range of 1 μm to 100 μm, and more preferably in the range of 1 μm to 50 μm. Although FIGS. 1A and 1B do not show, each of the source electrode 13 and the drain electrode 14 is formed on the substrate 11 in an extending manner so as to be electrically connected to the outside at a certain portion thereof.

Although FIGS. 1A and 1B do not show, the gate electrode, which is generally made of a noble metal, is to apply a potential to the source electrode 13 and the drain electrode 14. The gate electrode is disposed in the position other than the positions of the source electrode 13 and the drain electrode 14. The gate electrode is generally disposed on the substrate 11 or in the position other than the substrate 11. In the field effect transistor of the present invention, it is preferable to dispose the gate electrode above the source electrode 13 or the drain electrode 14.

Next, the manufacturing method of the single-walled carbon nanotube thin film 12 and the field effect transistor 1 is described. In the present invention, the single-walled carbon nanotube thin film 12 is used as a channel of the field effect transistor 1. The single-walled carbon nanotube thin film 12 is directly grown on the substrate 11, by the chemical vapor deposition method (CVD method), using particles of a nonmetallic material as growth nuclei, the nonmetallic material containing 500 mass ppm or less metallic impurities that contain a metal and its compounds.

Examples of the nonmetallic material used as the growth nuclei include: carbon materials such as diamond, diamond-like carbon, amorphous carbon particles, fullerene, graphite and carbon nanotube; or combined materials thereof. It is preferable to use any carbon materials that do not require the refining process, accordingly, it is preferable to use diamond, specifically, nanosized diamond, i.e., nanodiamond. The amount of the metallic impurities (including a metal and its compounds such as an oxide and a carbide) contained in the nonmetallic material should not affect product characteristics such as transparency and electrical conductivity in the application field. Thus, it should be 500 mass ppm or less, more preferably be 300 mass ppm or less.

Examples of diamond include: natural single crystal diamond; single crystal diamond by the static pressure method; polycrystalline diamond film by the CVD method; and diamond by the indirect explosion shock method and/or the direct explosion shock method. It is preferable to use the diamond synthesized by the indirect explosion shock method and/or the direct explosion shock method, and in particular, it is preferable to use the diamond synthesized by the direct explosion shock method, because it is most easily dispersed into the nano-size. The method for synthesizing diamond using the direct explosion shock method is described in, for example, WO 2007/001031. The diamond obtained by the above method is used as a dispersion obtained by the dispersion method such as bead milling, in which are dispersed diamond fine particles having a particle size of 50 nm or less, preferably 30 nm or less, more preferably 4 nm to 15 nm, the particle size at a cumulative frequency of 50% (hereinafter referred to as “50% particle size”) in the volume-based particle size distribution that is measured by dynamic light scattering. The nanodiamond particle dispersion is normally stable at ambient temperature. However, in order to obtain a more stable dispersion form in the organic medium, it is preferable to use hydrogenated nanodiamond that is obtained by processing diamond under the condition of temperature of 300° C. to 800° C. in a hydrogen atmosphere.

It is preferable to use the nonmetallic material as the growth nuclei by atomizing it using the mechanical method such as bead milling. In such a case, beads are also atomized by grinding and mixed into the nonmetallic material as the impurities. The single-walled carbon nanotubes obtained using the above nonmetallic material affect the film properties such as conductivity, transparency and haze. Therefore, the impurities such as a metal should be reduced as far as possible. The target content thereof is 500 mass ppm or less, preferably 300 mass ppm or less. The impurities derived from the metal containing metallic oxide is generally separated from the nonmetallic material after they are converted into water soluble salt under acidic or alkaline conditions using water as a solvent. At this time, the refining process may be accelerated by adding the heating process, if necessary.

Out of the nonmetallic materials as the growth nuclei that are necessary for the growth of the single-walled carbon nanotube thin film 12, the nanodiamond should have the particle size of 0.5 nm to 4 nm because it does not grow when having the particle size of 5 nm or more. Thus, when the nanodiamond having the particle size of more than 4 nm is used as the material for the growth nuclei, it should be treated so that the particle size falls within the above range. For this purpose, generally the following method is used: applying the nanodiamond particle dispersion liquid to the substrate so as to be dried; and subjecting the substrate to the heating treatment so as to obtain the desirable growth nuclei. As the conditions, in the case that no air is flown during the treatment, the treatment temperature is in the range of 500° C. to 800° C. and the treatment time is in the range of 1 minute to 60 minutes, and preferably, the treatment temperature is in the range of 500° C. to 700° C. and the treatment time is in the range of 1 minute to 30 minutes. In the case that air is flown during the treatment, the treatment temperature is in the range of 500° C. to 700° C. and the treatment time is in the range of 30 seconds to 30 minutes, and preferably, the treatment temperature is in the range of 500° C. to 600° C. and the treatment time is in the range of 30 seconds to 15 minutes. The particle size of the nanodiamond before the treatment may differ depending on the treatment temperature and the treatment time, however, in order to obtain the stable particle size of 0.4 nm to 4 nm after the heating treatment, with uniformity and high density as well as in a relatively short time, it is preferable that 50% particle size of the nanodiamond before the heating treatment is set to 50 nm or less, more preferably to 30 nm or less, and particularly to the range of 4 nm to 15 nm.

Growth gas (source gas), which is used as a carbon source when growing the single-walled carbon nanotubes, is any one of: hydrocarbon; alcohol; a mixture of hydrocarbon and alcohol; a mixture of hydrocarbon and water; a mixture of hydrocarbon, alcohol and water; and carbon monoxide. Examples of hydrocarbons include: saturated aliphatic hydrocarbons such as methane, ethane, propane and butane; unsaturated aliphatic hydrocarbons such as ethylene, propylene, butene and acetylene; alicyclic hydrocarbons such as cyclohexane and cyclohexene; and aromatic hydrocarbons such as benzene, toluene and xylene. It is preferable to use the unsaturated aliphatic hydrocarbon or the aromatic hydrocarbon, more preferably to use the unsaturated aliphatic hydrocarbon, and in particular, it is preferable to use acetylene. An oxygen-containing compound may be mixed so as to prevent unstable defects or amorphous carbon from generating during the growth of the single-walled carbon nanotubes. Examples of the oxygen-containing compounds include: water; alcohols; ketones; esters; and ethers. Examples of alcohols include: aliphatic alcohols such as methanol, ethanol, propyl alcohol, butanol, octyl alcohol, ethylene glycol, propylene glycol and glycerin; alicyclic alcohols such as cyclopentyl alcohol and cyclohexyl alcohol; glycol ethers such as ethoxy ethanol and propylene glycol monomethyl ether; aromatic alcohols such as phenol and cresol; ketones such as acetone, methyl ethyl ketone and methyl isobutyl ketone; carboxylic acid esters such as ethyl acetate, butyl acetate and propylene glycol monoacetate; and ethers such as ethyl ether and ethylene glycol dimethyl ether, which are used depending on the situation. However, it is preferable to use the water or the alcohols, and in particular, it is preferable to use the water or the ethanol. Also, the above oxygen-containing compounds except for the water can be used as the carbon source.

Hydrocarbon as the carbon source can be singly used, however, a mixture of hydrocarbon with the oxygen-containing compound can also be used. Also, the mixing ratio of hydrocarbon to the oxygen-containing compound may be fixed during the growth, however, it may be changed during the growth. In case of ethanol and acetylene, preferably the partial pressure ratio of ethanol to acetylene is in the range of 99.78:0.22 to 0:100. More preferably, the gas composition has the partial pressure ratio of ethanol to acetylene in the range of 97.09:2.91 to 0:100 at the initial growth stage, and after that, in the range of 100:0 to 99.55:0.45 at the growth stage (normal growth stage) of the single-walled carbon nanotubes. Thus, the partial pressure ratio of ethanol to acetylene at the normal growth stage is constantly greater than that at the initial growth stage. Also, acetylene is combustible and has a very high inflammability with an explosive limit in the range of 2.5 vol. % to 93 vol. %, which means a large explosive range and a very high explosion hazard. Thus, it is preferable to dilute acetylene with inert gas (dilution gas) having a high safety, and in particular, it is preferable to use argon. When acetylene is diluted with the dilution gas, the acetylene concentration in the dilution gas is set, normally, to 2.5 vol. % or less, preferably to 2 vol. % or less, so that the acetylene does not fall within the explosive limit.

When the single-walled carbon nanotube thin film 12 is grown, it is possible to use only the growth gas or to use multiple gasses such as the growth gas, the dilution gas and a carrier gas for efficiently carrying the growth gas to a sample region. As to pressure conditions during the growth, each partial pressure of the gas components or the total gas pressure as the sum of all of the partial pressures is often kept constant so as to grow the single-walled carbon nanotubes. However, the above pressures may be changed during the growth. When the gas pressure is constant, the pressure condition of the growth gas is in the range of 0.02 Pa to 20 kPa, preferably in the range of 0.1 Pa to 10 kPa. Also, by appropriately changing the pressure during the growth, it is possible to prepare high quality single-walled carbon nanotubes with high efficiency compared to the case in which the pressure is constant. As the pressure condition for the above, the pressure of the growth gas is in the range of 0.02 Pa to 20 kPa, preferably in the range of 0.1 Pa to 20 kPa at the initial growth stage and in the range of 0.02 Pa to 10 kPa at the normal growth stage. Thus, the pressure of the growth gas at the normal growth stage is smaller than the pressure at the initial growth stage. Furthermore, the total gas pressure in the CVD reactor is generally in the range of 0.02 Pa to 100 kPa, preferably in the range of 10 Pa to 20 kPa.

The total gas pressure as the sum of the partial pressures of the growth gas, the dilution gas and the carrier gas is often kept constant throughout the growing process. However, it may be changed, according to changing the pressure of the growth gas, normally within the range of 0.02 Pa to 100 kPa, preferably within the range of 10 Pa to 20 kPa. Also, at both of the initial growth stage and the normal growth stage, the above gas composition may be changed at the same time of changing the partial pressures or the total gas pressure as the sum of the partial pressures.

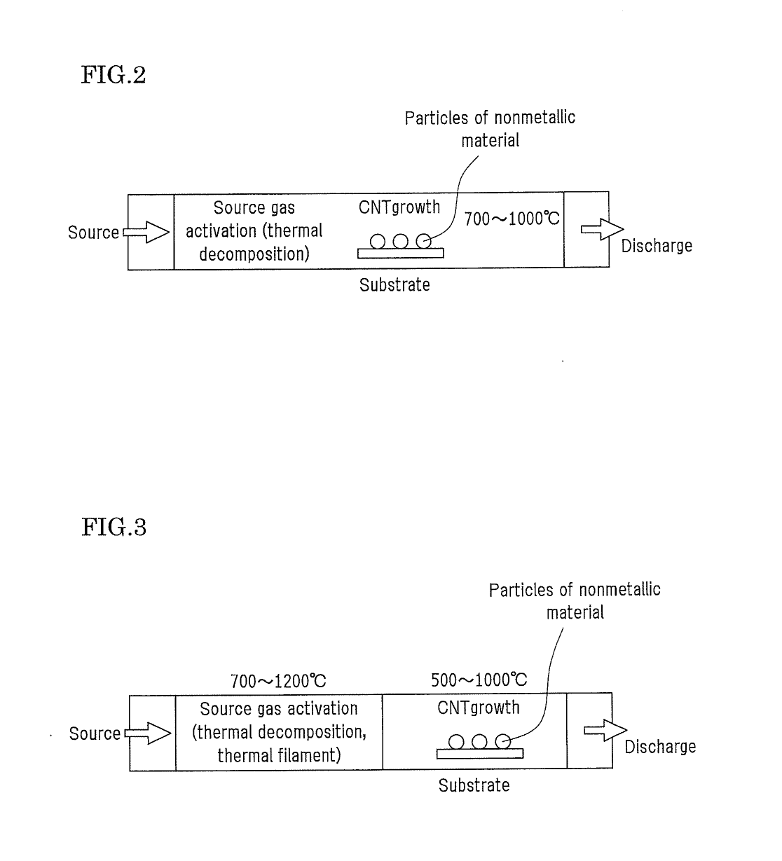

As the CVD reactor when nonmetallic material particles are used as the growth nuclei may be a conventionally used device as shown in FIG. 2, in which the following two temperatures are in a single temperature region: the temperature (growth gas activation temperature) upstream of the vicinity of the substrate including nonmetallic material particles (for example, the substrate on which the growth nuclei are formed); and the temperature in the vicinity of the substrate (CNT growth temperature). The nonmetallic material particles do not have the effect to accelerate the decomposition of the growth gas unlike metal catalysts such as iron and cobalt, which are conventionally used. Therefore, in order to grow the single-walled carbon nanotubes from the nonmetallic material particles, it is necessary to perform the treatment at the temperature not less than the temperature that allows the growth gas to decompose spontaneously. The optimum temperature region may differ depending on the kind and the mixed composition of the used growth gas, however, it is normally in the range of 700° C. to 1000° C.

In order to obtain higher quality and more pure single-walled carbon nanotubes with high efficiency, it is possible and preferable to use a CVD reactor with multiple temperature zones as shown in FIG. 3, in which the growth gas activation temperature and the single-walled carbon nanotube growth temperature are separately set. By the use of this reactor, it is possible to separately control the surface diffusion on the growth nuclei and the formation process of the single-walled carbon nanotubes, whereby obtaining higher quality and more pure single-walled carbon nanotubes with high efficiency. In this case also, the optimum temperature region may differ depending on the kind, the partial pressure and the mixed composition of the used growth gas. However, the growth gas activation temperature on the upstream side is in the range of 700° C. to 1200° C., preferably in the range of 700° C. to 900° C. while the temperature in the vicinity of the substrate is in the range of 500° C. to 1000° C., preferably in the range of 600° C. to 850° C. At this time, it is possible to obtain results suitable for the growth with high quality and high efficiency under the condition that the temperature on the upstream side is set to be sufficiently higher than the thermal decomposition temperature of the growth gas and the temperature in the vicinity of the substrate is set to be constantly equal to or higher than the thermal decomposition temperature of the growth gas.

The nonmetallic material particles containing 500 mass ppm or less metallic impurities are used as the growth nuclei for the single-walled carbon nanotube thin film 12 obtained as described above. Thus, the metallic impurities are considerably reduced and the process for removing/refining metals is not required, which is necessary for the case in which the conventional metal material is used as the growth nuclei.

When the single-walled carbon nanotube thin film 12 is directly grown on the substrate 11 composed of a highly heat-resistant inorganic compound or the like, the conventional dispersion process is not needed. It is possible to use the layer of the single-walled carbon nanotube thin film 12 as is, which is grown on the substrate 11. When the substrate composed of a non-heat-resistant organic compound such as a resin film is used as the substrate 11, the single-walled carbon nanotube thin film 12 can be formed by preparing the single-walled carbon nanotubes using a method similar to the CoMoCAT method and fixing the single-walled carbon nanotubes on the film by the cooling process. Also, it is possible to prepare the single-walled carbon nanotube thin film 12 on the substrate 11 composed of any organic compound by synthesizing the single-walled carbon nanotubes on the substrate composed of a highly heat-resistant inorganic compound or the like and transferring the synthesized single-walled carbon nanotubes on the substrate 11 composed of the organic compound.

In the present invention, the single-walled carbon nanotube thin film 12 is grown by the chemical vapor deposition method using the nonmetallic material particles as the growth nuclei. Thus, the dispersion process and the metal removing/refining process, which are needed in the conventional art, are not needed. Therefore, it is possible to prevent the single-walled carbon nanotube thin film 12 from having defects or being cut due to these processes.

After the growth of the single-walled carbon nanotubes on the substrate 11, the single-walled carbon nanotube thin film 12 is formed so as to have a predetermined shape by patterning the single-walled carbon nanotubes by the photolithography and the etching. After that, the Ti layer and the Au layer are formed on the substrate 11 and the single-walled carbon nanotube thin film 12 using the method such as the vacuum deposition, the electro beam deposition or the sputtering. Then, an insulating thin film made of silicon dioxide is formed. After the layered structure of Ti/Au/silicon dioxide is formed on the substrate 11 so as to cover the single-walled carbon nanotube thin film 12, it is patterned by the photolithography and the etching so as to form the source electrode 13, the drain electrode 14 and the coating layer. After that, the gate electrode is disposed in a certain position. Thus, the field effect transistor 1 of the present invention is formed.

### Example 1

(a) Preparation of Single-Walled Carbon Nanotube Thin Film (with High Density)

On a 1 cm square thermally oxidized silicon substrate as the substrate 11, which was made by forming the silicon oxide layers 11b respectively on the surface and the rear surface of the silicon substrate 11a by oxidation, ethanol dispersion liquid containing 2.0 mass % hydrogenated nanodiamond (trade name: “Ustalla (registered trade mark) Type C”, manufactured by Nippon Kayaku Co., Ltd.) having the particle size distribution of 5 nm to 15 nm and the metallic impurities of 100 mass ppm was applied by the spin coating method so as to obtain a substrate on which the hydrogenated nanodiamond was applied. The spin coating conditions were as follows: Step I: at 300 rpm, for 30 seconds; Step II: slope, for 30 seconds; and Step III: 1000 rpm, for 60 seconds. Sequentially, the substrate on which the hydrogenated nanodiamond had been applied was placed in a heating furnace so as to be subjected to the heating treatment in the atmosphere at 600° C. for 15 minutes, thus the hydrogenated nanodiamond growth nuclei having the particle size of 0.5 nm to 4 nm were obtained. The substrate was placed in the CVD reactor handling the multiple temperature conditions as shown in FIG. 3 and the single-walled carbon nanotube thin film 12 was obtained by flowing, for 30 minutes, gas composed of 10 sccm acetylene (diluted with argon as dilution gas to 2 vol. %) as the growth gas and 10 sccm argon-hydrogen carrier gas under the condition that the temperature on the upstream side was 850° C., the temperature in the vicinity of the substrate was 780° C. and the pressure was 500 Pa. In the single-walled carbon nanotube thin film 12 in Example 1, no impurity derived from the metal could be detected by a scanning electron microscope and an energy-dispersive X-ray spectrometer (SEM-EDS). Thus, it was seen that a metal-free single-walled carbon nanotube thin film was prepared.

When the obtained single-walled carbon nanotube thin film was evaluated using a Raman spectrometer, the ratio I(G)/I(Si) was 1.2, and the ratio I(G)/I(D) was 4.8.

### (b) Manufacture of Field Effect Transistor (FET)

Next, the field effect transistor 1 in Example 1 was manufactured according to the procedures stated below, using the single-walled carbon nanotube thin film 12 grown on the thermally oxidized silicon substrate.

(1) Resist Coating Process: The single-walled carbon nanotube thin film, on which 1,1,1,3,3,3-hexamethyldisilazane (HMDS) serving as a surfactant was applied by a spin coater, was baked (at about 120° C., for 2 minutes). Then, photoresist (trade name: “OFPR (registered trade mark) -800LB”, manufactured by TOKYO OHKA KOGYO CO., LTD) was applied, by the spin coater, on the single-walled carbon nanotube thin film so as to form a photoresist film. Finally, the work (i.e., the thermally oxidized silicon substrate on which the single-walled carbon nanotube thin film and the photoresist film were formed) was baked (at about 90° C., for 5 minutes).

(2) Photolithography Process: A pattern was created using Cad data by exposing the sample to light using a maskless exposure device (trade name: “DL-1000”, manufactured by Nanosystem Solutions, Inc.).

(3) Development Process: The work after being subjected to photolithography was immersed in a developer (trade name: “NMD-3”, manufactured by TOKYO OHKA KOGYO CO., LTD., which is aqueous solution containing 2.38 mass % tetramethyl ammonium hydroxide) for 90 seconds, and then was rinsed with ultrapure water.

(4) Channel and Alignment marker: The CNT thin film that was not protected by the photoresist film after the development was etched by a reactive ion etching device (trade name: “RIE-10NR”, manufactured by Samco Inc.), thus a channel and an alignment marker were prepared at the same time from the CNT thin film.

(5) Lift-off Process: The work was immersed in 1-methyl-2-pyrrolidone as a photoresist peeling liquid so as to lift (peel) the photoresist film off. The time for immersion was appropriately adjusted depending on the film thickness of the photoresist film or the film thickness of the vapor coating material.

(6) Electrode Producing Process: On exposed portions of the upper surfaces of the single-walled carbon nanotube thin film 12 and the thermally oxidized silicon substrate, the following were formed by the vacuum deposition, the electro beam deposition or the sputtering: Ti layer having the layer thickness of 5 nm; Au layer having the layer thickness of 45 nm; and alumina (Al₂O₃) layer having the layer thickness of 30 nm, thus were obtained a channel 10 μm wide and 5 μm long, a source electrode and a drain electrode. A back gate electrode was provided on the rear surface of the substrate through the silicon oxide film having the thickness of 300 nm on the surface of the thermally oxidized silicon substrate, thus a field effect transistor was obtained.

### Example 2

(a) Preparation of Single-Walled Carbon Nanotube Thin Film (with Low Density)

On a 1 cm square thermally oxidized silicon substrate as the substrate 11, which was made by forming the silicon oxide layers 11b respectively on the surface and the rear surface of the silicon substrate 11a by oxidation, ethanol dispersion liquid containing 2.0 mass % hydrogenated nanodiamond (trade name: “Ustalla (registered trade mark) Type C”, manufactured by Nippon Kayaku Co., Ltd.) having the particle size distribution of 5 nm to 15 nm and the metallic impurities of 100 mass ppm was applied by the spin coating method so as to obtain a substrate on which the hydrogenated nanodiamond was applied. The spin coating conditions were the same as those in Example 1. Sequentially, the substrate on which the hydrogenated nanodiamond had been applied was placed in the heating furnace so as to be subjected to the heating treatment in the atmosphere at 600° C. for 15 minutes, thus the hydrogenated nanodiamond growth nuclei having the particle size of 0.5 nm to 4 nm were obtained. The substrate was placed in the CVD reactor handling the multiple temperature conditions as shown in FIG. 3 and the single-walled carbon nanotube thin film 12 was obtained by flowing, for 30 minutes, gas composed of 10 sccm acetylene (diluted with argon as dilution gas to 2 vol. %) as the growth gas and 10 sccm argon-hydrogen carrier gas under the condition that the temperature on the upstream side was 850° C., the temperature in the vicinity of the substrate was 780° C. and the pressure was 500 Pa.

When the obtained single-walled carbon nanotube thin film was evaluated using the Raman spectrometer, the ratio I(G)/I(Si) was 0.75, and the ratio I(G)/I(D) was 5.0. Thus, it was confirmed that the obtained single-walled carbon nanotube thin film had the density lower than that obtained in Example 1(a). Using the obtained single-walled carbon nanotube thin film, a field effect transistor was manufactured in the same way as Example 1.

### [Comparative Example 1] Preparation of Single-Walled Carbon Nanotube Thin Film (with High Density) Grown from Co

On a 1 cm square crystal substrate, cobalt was vapor-deposited so as to obtain a crystal substrate to which was attached a cobalt film having the thickness of 0.5 nm that serves as the growth nuclei. The crystal substrate with the cobalt film was placed in the heating furnace so as to be subjected to the heating treatment in the atmosphere at 600° C. for 10 minutes. Next, the crystal substrate with the cobalt film was placed in the CVD reactor handling the constant temperature condition and the single-walled carbon nanotube thin film was obtained by flowing, for 30 minutes, gas composed of 6 sccm ethanol as the growth gas and 14 sccm argon-hydrogen carrier gas under the condition that the temperature on the upstream side was 850° C., the temperature in the vicinity of the substrate was 780° C. and the pressure was 250 Pa.

When the obtained single-walled carbon nanotube thin film was evaluated using the Raman spectrometer, the ratio I(G)/I(Si) was 1.8, and the ratio I(G)/I(D) was 5.0. Thus, it was confirmed that the high quality single-walled carbon nanotube thin film was obtained with high efficiency (yield), which was substantially equivalent to that obtained in Example 1(a). Using the obtained single-walled carbon nanotube thin film, a field effect transistor was manufactured in the same way as Example 1.

### [Comparable Example 2] Preparation of Single-Walled Carbon Nanotube Thin Film (with Low Density) Grown from Co

On a 1 cm square crystal substrate, cobalt was vapor-deposited so as to obtain a crystal substrate to which was attached a cobalt film having the thickness of 0.5 nm that serves as the growth nuclei. The crystal substrate with the cobalt film was placed in the heating furnace so as to be subjected to the heating treatment in the atmosphere at 600° C. for 10 minutes. Next, the crystal substrate with the cobalt film was placed in the CVD reactor handling the constant temperature condition and the single-walled carbon nanotube thin film was obtained by flowing, for 30 minutes, gas composed of 10 sccm acetylene as the growth gas and 10 sccm argon-hydrogen carrier gas under the condition that the temperature on the upstream side was 850° C., the temperature in the vicinity of the substrate was 780° C. and the pressure was 500 Pa.

When the obtained single-walled carbon nanotube thin film was evaluated using the Raman spectrometer, the ratio I(G)/I(Si) was 0.6, and the ratio I(G)/I(D) was 4.0. Thus, it was confirmed that the high quality single-walled carbon nanotube thin film was obtained with high efficiency (yield), which was substantially equivalent to that obtained in Example 2(a). Using the obtained single-walled carbon nanotube thin film, a field effect transistor was manufactured in the same way as Example 1.

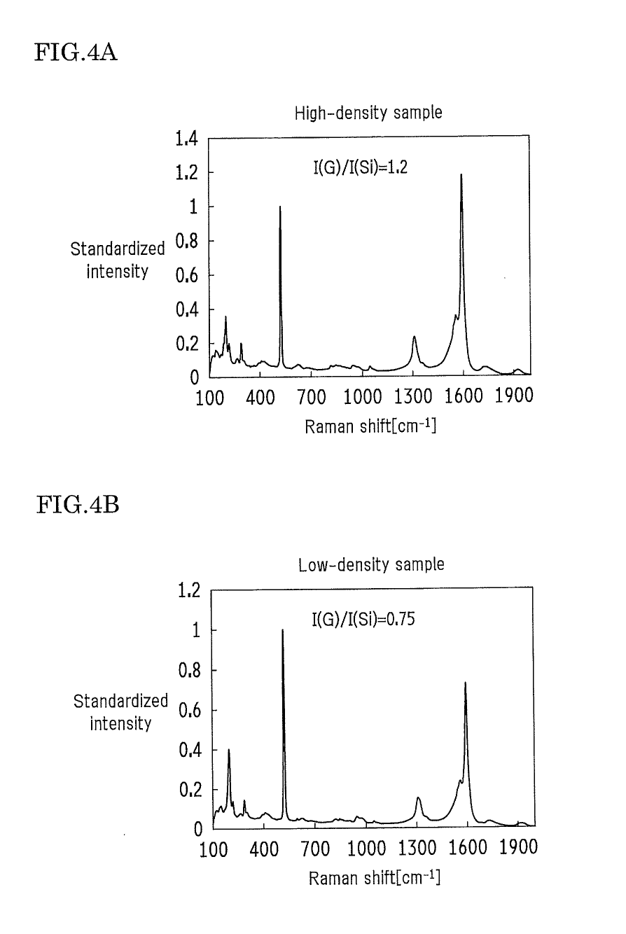

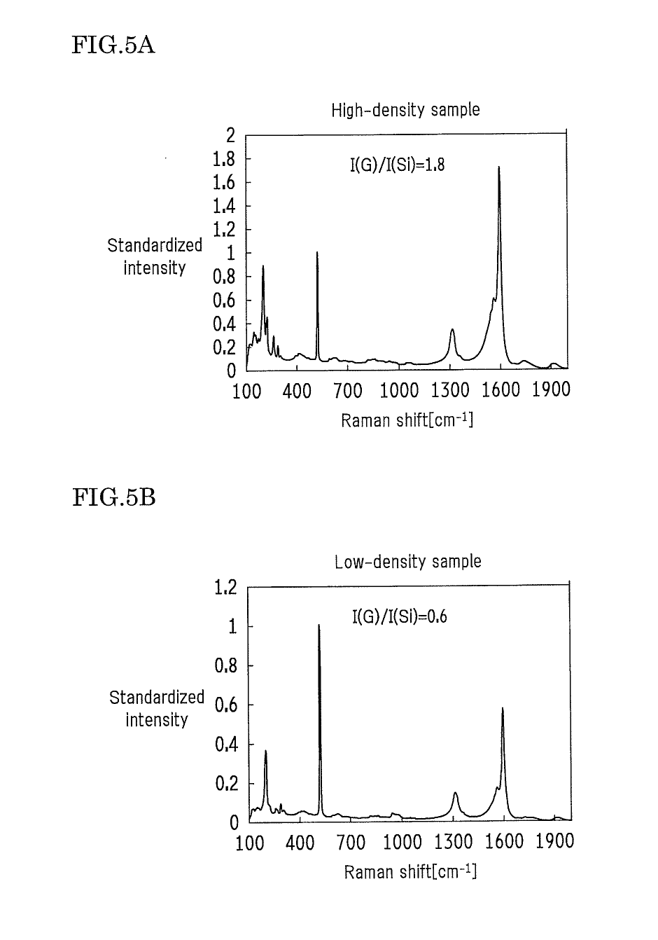

FIGS. 4A and 4B are graphs showing respectively the Raman spectrums of the high-density sample and the low-density sample of the single-walled carbon nanotube thin films grown from nanodiamond in Examples 1 and 2. FIGS. 5A and 5B are graphs showing respectively the Raman spectrums of the high-density sample and the low-density sample of the single-walled carbon nanotube thin films grown from Co in Comparative Examples 1 and 2.

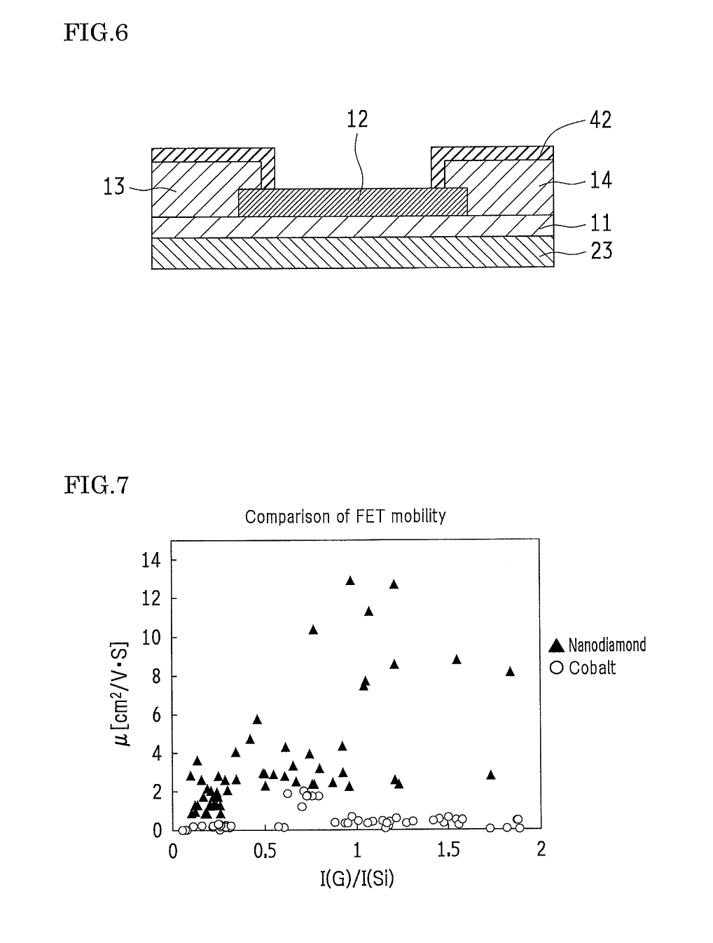

Next, apart from Examples 1 and 2, multiple single-walled carbon nanotube thin films were prepared by being grown from nanodiamond under conditions different from those in Examples 1 and 2. Also, apart from Comparative Examples 1 and 2, multiple single-walled carbon nanotube thin films were prepared by being grown from cobalt under conditions different from those in Comparative Examples 1 and 2. Furthermore, the respective samples of field effect transistors as shown FIG. 6 were manufactured from the above. The sample field effect transistor as shown in FIG. 6 has a configuration similar to that in FIG. 1A. However, in this configuration, a back gate electrode as a gate electrode 23 is formed on the rear surface of the substrate 11, and the coating layer 42 is formed on each surface of the source electrode 12 and the drain electrode 13. Gate characteristics of the respective samples were measured using semiconductor parameters so as to calculate a carrier mobility μ. In order to calculate the carrier mobility μ, the following Mathematical 1 was used, where L_{ch} represents the channel length, W_{ch} represents the channel width, V_{sd} represents the source-drain voltage, C_{g} represents the gate capacitance, I_{sd} represents the source-drain current and V_{g} represents the gate voltage.

$$\begin{matrix}[ Mathematical 1 ] & \\ μ = ( \frac{{L}_{ch}}{{W}_{ch}} ) ( \frac{1}{{V}_{sd}} ) ( \frac{1}{{C}_{g}} ) ( \frac{{dI}_{sd}}{{dV}_{g}} ) & ( 1 )\end{matrix}$$

Here, the gate capacitance C_{g} can be obtained by the following Mathematical 2 using the permittivity (εₒₓ) of the insulating film between the gate and the carrier and the thickness (dₒₓ) of the insulating film (e.g., silicon oxide film).

$$\begin{matrix}[ Mathematical 2 ] & \\ {C}_{g} = \frac{{ɛ}_{ox}}{{d}_{ox}} & ( 2 )\end{matrix}$$

FIG. 7 is a graph plotted with each measured ratio I(G)/I(Si) as abscissa and each calculated carrier mobility μ [cm²/V·s] as ordinate regarding the above multiple samples. In this graph, the samples in the group in which nanodiamond was used as the growth nuclei are plotted with black triangles while the samples in the group in which cobalt was used as the growth nuclei are plotted with outlined circles. In all areas of the ratio I(G)/I(Si), the carrier mobility of each sample in the group in which nanodiamond was used as the growth nuclei is larger, by one digit or more, than the carrier mobility of each sample in the group in which cobalt was used as the growth nuclei. Thus, it can be seen that good FET characteristics are realized.

It can be seen, from the results of the multiple samples, that even when the ratio I(G)/I(Si) is substantially the same, the corresponding carrier mobility considerably differs. Therefore, it is possible to consider that the following manufacturing method contributes to improvement of performance of the field effect transistor 1: growing the single-walled carbon nanotube thin film, by the chemical vapor deposition method, using particles of a nonmetallic material as the growth nuclei, the nonmetallic material containing 500 mass ppm or less metallic impurities that contain a metal and its compounds.

### Example 3

### (a) Preparation of (Long) Single-Walled Carbon Nanotube Thin Film

On a 1 cm square thermally oxidized silicon substrate as the substrate 11, which was made by forming the silicon oxide layers 11b respectively on the surface and the rear surface of the silicon substrate 11a by oxidation, ethanol dispersion liquid containing 2.0 mass % hydrogenated nanodiamond (trade name: “Ustalla (registered trade mark) Type C”, manufactured by Nippon Kayaku Co., Ltd.) having the particle size distribution of 5 nm to 15 nm and the metallic impurities of 100 mass ppm was applied in the amount of 20 μl by the falling-drop method so as to obtain a substrate on which was applied the hydrogenated nanodiamond having the layer thickness corresponding to the thickness of 200 particles. Here, the particle layer thickness was calculated using the average particle size, the mass concentration and the specific gravity of diamond. Sequentially, the substrate on which the hydrogenated nanodiamond had been applied was placed in the heating furnace so as to be subjected to the heating treatment in the atmosphere at 600° C. for 15 minutes, thus the hydrogenated nanodiamond growth nuclei having the particle size of 0.5 nm to 4 nm were obtained. The substrate was placed in the CVD reactor handling the multiple temperature conditions as shown in FIG. 3 and the single-walled carbon nanotube thin film 12 was obtained by flowing, for 2 minutes, gas composed of 10 sccm acetylene (diluted with argon as dilution gas to 2 vol. %) as the growth gas and 10 sccm argon-hydrogen carrier gas, and sequentially by flowing, for 28 minutes, gas composed of 2 sccm acetylene (diluted with argon as dilution gas to 2 vol. %) and 18 sccm argon-hydrogen carrier gas, under the condition that the temperature on the upstream side was 850° C., the temperature in the vicinity of the substrate was 780° C. and the pressure was 500 Pa (growth time: 30 minutes).

When the obtained single-walled carbon nanotube thin film was evaluated using the Raman spectrometer, the ratio I(G)/I(Si) was 2.88, and the ratio I(G)/I(D) was 5.75. Using the obtained single-walled carbon nanotube thin film, a field effect transistor was manufactured in the same way as Example 1.

### Example 4

### (a) Preparation of (Long) Single-Walled Carbon Nanotube Thin Film

On a 1 cm square thermally oxidized silicon substrate as the substrate 11, which was made by forming the silicon oxide layers 11b respectively on the surface and the rear surface of the silicon substrate 11a by oxidation, ethanol dispersion liquid containing 2.0 mass % hydrogenated nanodiamond (trade name: “Ustalla (registered trade mark) Type C”, manufactured by Nippon Kayaku Co., Ltd.) having the particle size distribution of 5 nm to 15 nm and the metallic impurities of 100 mass ppm was applied in the amount of 10 μl by the falling-drop method so as to obtain a substrate on which was applied the hydrogenated nanodiamond having the layer thickness corresponding to the thickness of 100 particles. Sequentially, the substrate on which the hydrogenated nanodiamond had been applied was placed in the heating furnace so as to be subjected to the heating treatment in the atmosphere at 600° C. for 15 minutes, thus the hydrogenated nanodiamond growth nuclei having the particle size of 0.5 nm to 4 nm were obtained. The substrate was placed in the CVD reactor handling the multiple temperature conditions as shown in FIG. 3 and the single-walled carbon nanotube thin film 12 was obtained by flowing, for 2 minutes, gas composed of 10 sccm acetylene (diluted with argon as dilution gas to 2 vol. %) as the growth gas and 10 sccm argon-hydrogen carrier gas, and sequentially by flowing, for 88 minutes, gas composed of 2 sccm acetylene (diluted with argon as dilution gas to 2 vol. %) and 18 sccm argon-hydrogen carrier gas, under the condition that the temperature on the upstream side was 850° C., the temperature in the vicinity of the substrate was 780° C. and the pressure was 500 Pa (growth time: 90 minutes).

When the obtained single-walled carbon nanotube thin film was evaluated using the Raman spectrometer, the ratio I(G)/I(Si) was 0.94, and the ratio I(G)/I(D) was 4.76. Using the obtained single-walled carbon nanotube thin film, a field effect transistor was manufactured in the same way as Example 1.

### Example 5

Field effect transistors were manufactured by a method similar to that in Example 3 except that the use amount of the ethanol dispersion liquid of hydrogenated nanodiamond was changed, respectively, to 10 μl and to 5 μl so that the obtained hydrogenated nanodiamond in each case had the layer thickness corresponding to the thickness of 100 particles and 50 particles.

Also, a field effect transistor was manufactured by a method similar to that in Example 3 except that the period of time for flowing the growth gas, which was composed of 2 sccm acetylene (diluted with argon as dilution gas to 2 vol. %) and 18 sccm argon-hydrogen carrier gas, was changed to 58 minutes so that the growth time was changed to 60 minutes.

Also, field effect transistors were manufactured by a method similar to that in Example 3 except that the period of time for flowing the gas, which was composed of 2 sccm acetylene (diluted with argon as dilution gas to 2 vol. %) and 18 sccm argon-hydrogen carrier gas, was changed to 58 minutes so that the growth time was changed to 60 minutes, and that the use amount of the ethanol dispersion liquid of hydrogenated nanodiamond was changed, respectively, to 10 μl and to 5 μl so that the obtained hydrogenated nanodiamond in each case had the layer thickness corresponding to the thickness of 100 particles and 50 particles.

A field effect transistor was manufactured by a method similar to that in Example 4 except that the use amount of the ethanol dispersion liquid of hydrogenated nanodiamond was changed to 5 μl so that the obtained hydrogenated nanodiamond had the layer thickness corresponding to the thickness of 50 particles.

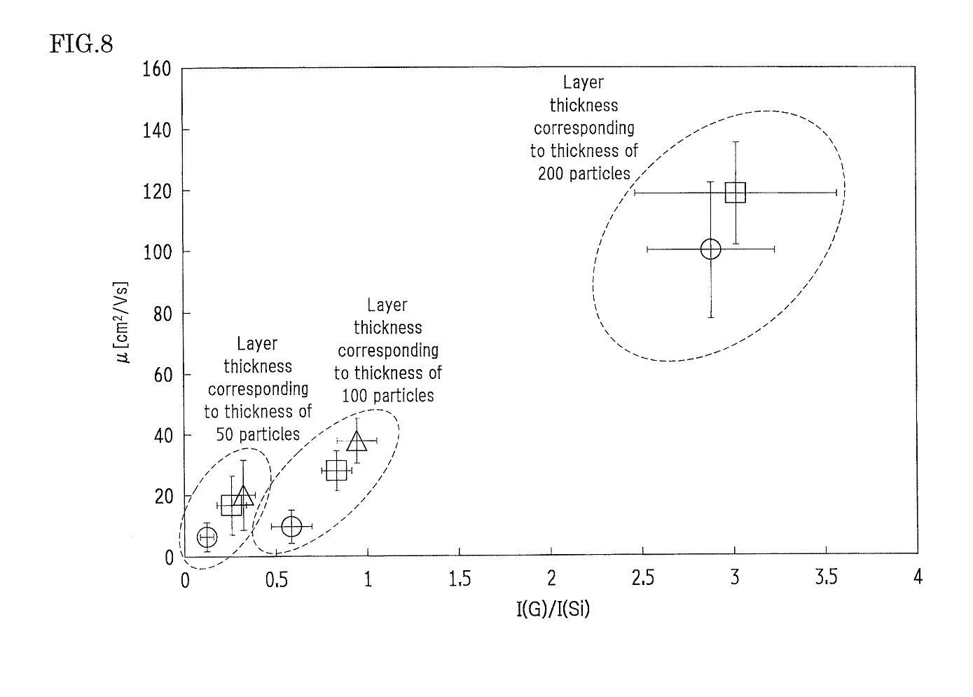

FIG. 8 is a graph plotted with each measured ratio I(G)/I(Si) as abscissa and each carrier mobility μ as ordinate regarding the field effect transistors 1 in Examples 3, 4 and 5. In FIG. 8, the field effect transistors in which the growth time of the single-walled carbon nanotube thin film 12 was 30 minutes are indicated as circles, the transistors in which the growth time of the single-walled carbon nanotube thin film 12 was 60 minutes are indicated as quadrangles, and the transistors in which the growth time of the single-walled carbon nanotube thin film 12 was 90 minutes are indicated as triangles. Also, the transistors in which hydrogenated nanodiamond had the layer thickness corresponding to the thickness of 50 particles, 100 particles and 200 particles are respectively indicated using the dashed lines in the graph. As can clearly be seen from FIG. 8, there is a tendency that the carrier mobility μ increases as the thickness of hydrogenated nanodiamond applied on the substrate increases and furthermore as the growth time increases.

### Example 6

### (a) Preparation of (Long) Single-Walled Carbon Nanotube Thin Film Using Alcohol Gas

On a 1 cm square thermally oxidized silicon substrate as the substrate 11, which was made by forming the silicon oxide layers 11b respectively on the surface and the rear surface of the silicon substrate 11a by oxidation, ethanol dispersion liquid containing 2.0 mass % hydrogenated nanodiamond (trade name: “Ustalla (registered trade mark) Type C”, manufactured by Nippon Kayaku Co., Ltd.) having the particle size distribution of 5 nm to 15 nm and the metallic impurities of 100 mass ppm was applied in the amount of 45 μl by the spin coating method so as to obtain a substrate on which the hydrogenated nanodiamond was applied. The spin coating conditions were the same as those in Example 1.

Sequentially, the substrate on which the hydrogenated nanodiamond had been applied was placed in the heating furnace so as to be subjected to the heating treatment in the atmosphere at 600° C. for 15 minutes, thus the hydrogenated nanodiamond growth nuclei having the particle size of 0.5 nm to 4 nm were obtained. The substrate was placed in the CVD reactor handling the multiple temperature conditions as shown in FIG. 3 and the single-walled carbon nanotube thin film 12 was obtained by flowing, for 2 minutes, gas composed of: 9 sccm acetylene (diluted with argon as dilution gas to 2 vol. %) and 1 sccm ethanol gas as the growth gas; and 10 sccm argon-hydrogen carrier gas, and sequentially by flowing, for 58 minutes, gas composed of 10 sccm ethanol gas and 10 sccm argon-hydrogen carrier gas, under the condition that the temperature on the upstream side was 850° C., the temperature in the vicinity of the substrate was 780° C. and the pressure was 500 Pa.

When the obtained single-walled carbon nanotube thin film was evaluated using the Raman spectrometer, the ratio I(G)/I(Si) was 0.0694, and the ratio I(G)/I(D) was 1.87. Using the obtained single-walled carbon nanotube thin film, a field effect transistor was manufactured in the same way as Example 1.

### Example 7

### (a) Preparation of (Long) Single-Walled Carbon Nanotube Thin Film Using Alcohol Gas

On a 1 cm square thermally oxidized silicon substrate as the substrate 11, which was made by forming the silicon oxide layers 11b respectively on the surface and the rear surface of the silicon substrate 11a by oxidation, ethanol dispersion liquid containing 2.0 mass % hydrogenated nanodiamond (trade name: “Ustalla (registered trade mark) Type C”, manufactured by Nippon Kayaku Co., Ltd.) having the particle size distribution of 5 nm to 15 nm and the metallic impurities of 100 mass ppm was applied in the amount of 20 μl by the falling-drop method. Sequentially, the substrate on which the hydrogenated nanodiamond had been applied was placed in the heating furnace so as to be subjected to the heating treatment in the atmosphere at 600° C. for 15 minutes, thus the hydrogenated nanodiamond growth nuclei having the particle size of 0.5 nm to 4 nm were obtained. The substrate was placed in the CVD reactor handling the multiple temperature conditions as shown in FIG. 3 and the single-walled carbon nanotube thin film 12 was obtained by flowing, for 30 minutes, gas composed of 0.8 sccm ethanol gas as the growth gas and 19.2 sccm argon-hydrogen carrier gas, under the condition that the temperature on the upstream side was 850° C., the temperature in the vicinity of the substrate was 780° C. and the pressure was 5 kPa.

When the obtained single-walled carbon nanotube thin film was evaluated using the Raman spectrometer, the ratio I(G)/I(Si) was 0.153, and the ratio I(G)/I(D) was 12.8. Using the obtained single-walled carbon nanotube thin film, a field effect transistor was manufactured in the same way as Example 1.

### Second Embodiment

Next, as the second embodiment of the present invention, description will be given on a case in which the field effect transistor 1 according to the first embodiment, which serves as a transducer, is used in a chemical sensor for detecting chemical substances and the like.

The field effect transistor 1 of the present invention can be manufactured as a device without requiring any complicated process. Furthermore, the sensor including the field effect transistor 1 as a transducer has not only a good chemical stability but also a very high sensitivity. Therefore, it is particularly suitable for use in a chemical sensor to detect the negligible quantity of chemical substances and the like. As the transducer, the field effect transistor 1 can be used as is for detecting ions in the water or sensing the pH value of the solution.

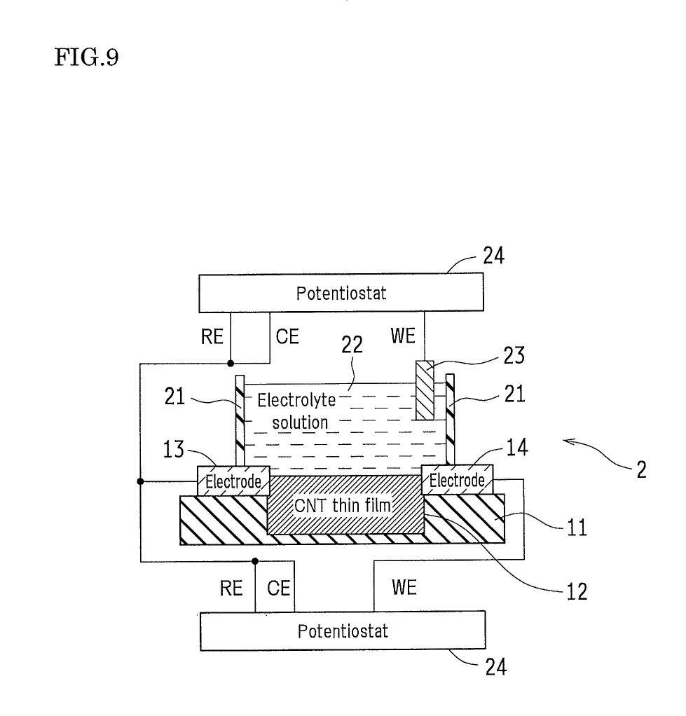

FIG. 9 is a schematic diagram showing a configuration of a sensor 2 according to this embodiment. The sensor 2 is configured, for example, by attaching a pool 21 made of silicone rubber on the field effect transistor 1 shown in FIGS. 1A and 1B, filling the pool 21 with a solution 22 containing an analyte, immersing a gate electrode 23 of the field effect transistor 1 in the solution 22, and connecting a bi-potentiostat 24 to the respective electrodes 13, 14 and 23 of the field effect transistor 1.

### Example 8

The sensor 2 shown in FIG. 9 was manufactured using: the field effect transistor 1 in Example 1; a pool made of silicone rubber as the pool 21; and a bi-potentiostat (trade name: “HZ-7000”) manufactured by HOKUTO DENKO CORPORATION as the bi-potentiostat 24.

### Example 9

The sensor was manufactured in the same way as Example 8 except that the field effect transistor 1 in Example 2 was used in place of the field effect transistor 1 in Example 1.

### Comparative Example 3

The sensor was manufactured in the same way as Example 8 except that the field effect transistor 1 in Comparative Example 1 was used in place of the field effect transistor 1 in Example 1.

### Comparative Example 4

The sensor was manufactured in the same way as Example 8 except that the field effect transistor 1 in Comparative Example 2 was used in place of the field effect transistor 1 in Example 1.

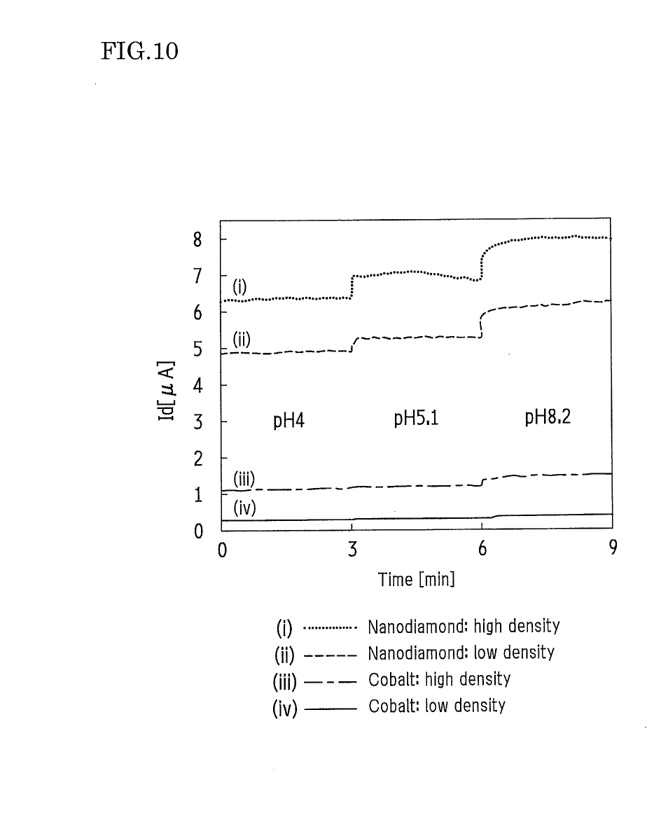

FIG. 10 is a graph showing pH measurement results of an electrolyte solution that is the solution 22 using the sensors 2 in Examples 8 and 9 and the sensors in Comparative Examples 3 and 4. In the pH measurement, the pH value of the solution 22 was changed using, as buffer solutions, a phthalate buffer solution (pH 4), a phosphate buffer solution (pH 6.8) and a borate buffer solution (pH 9) (all manufactured by HORIBA, Ltd.). Each concentration of these buffer solutions was adjusted to 10 mM by being diluted with the ultrapure water. Thus, change in each source-drain current I_{sd} with respect to time was recorded. The measurement was conducted by applying the gate voltage (in this case, top gate voltage) V_{g}=−0.1 V and the source-drain voltage V_{sd}=0.1 V.

First, the pool 21 made by cutting out silicone rubber was disposed on the substrate 11 so as to serve as a partition wall for keeping the solution on the substrate. Then, the pool 21 was well rinsed by the phthalate buffer solution (pH 4). After that, 50 μl phthalate buffer solution (pH 4) was dripped into the pool 21. The recording of the source-drain current was started, and three minutes later, 50 μl phosphate buffer solution (pH 6.8) was dripped and sufficiently stirred. Another three minutes later, 50 μl borate buffer solution (pH 9) was dripped and sufficiently stirred. The change in the source-drain current value I_{sd} was recorded for further three minutes.

In FIG. 10, the measurement result measured by the sensor 2 in Example 8 is indicated by the dotted line (i), in which was used, as the transducer, a high density sample of the single-walled carbon nanotube thin film 12 grown from nanodiamond as the growth nuclei in the field effect transistor 1 in Example 1. The measurement result measured by the sensor 2 in Example 9 is indicated by the dashed line (ii), in which was used, as the transducer, a low density sample of the single-walled carbon nanotube thin film 12 grown from nanodiamond as the growth nuclei in the field effect transistor 1 in Example 2. The measurement result measured by the sensor 2 in Comparative Example 3 is indicated by the dashed-dotted line (iii), in which was used, as the transducer, a high density sample of the single-walled carbon nanotube thin film 12 grown from cobalt as the growth nuclei in Comparative Example 1. The measurement result measured by the sensor 2 in Comparative Example 4 is indicated by the solid line (iv), in which was used, as the transducer, a low density sample of the single-walled carbon nanotube thin film 12 grown from cobalt as the growth nuclei in Comparative Example 2.

As can be seen from FIG. 10, when the same volumes of buffer solutions are sequentially dripped so that the pH value of the solution 22 is changed from 4 to 5.1 and further to 8.2, it is observed that the current value changes in a stepwise manner during dripping the buffer solutions. A large amount of change is likely to be observed in the results (i) and (ii) respectively corresponding to Examples 8 and 9 in which nanodiamond was used as the growth nuclei. On the other hand, a small amount of change is likely to be observed in the results (iii) and (iv) respectively corresponding to Comparative Examples 3 and 4 in which cobalt was used as the growth nuclei. Accordingly, it can be seen that the sensitivity of the sensor is improved by the single-walled carbon nanotube thin film 12 using nanodiamond as the nonmetallic material containing 500 mass ppm or less metallic impurities that contain a metal and its compounds, compared to the single-walled carbon nanotube thin film using the metal catalyst.

### Third Embodiment

Next, as the third embodiment of the present invention, a biosensor will be described, in which the field effect transistor 1 of the first embodiment is used as the transducer.

The field effect transistor 1 of the present invention can be manufactured as a device without requiring any complicated process. Furthermore, the sensor including the field effect transistor 1 as the transducer has not only a good chemical stability but also a very high sensitivity. Therefore, it is particularly suitable for use in a biosensor for detecting a minor component. By using the field effect transistor 1 as the transducer, and by modifying the channel with a specific substance that specifically interacts with the analyte, the analyte as a target can be selectively detected with a high sensitivity. Examples of the analytes include cells, microorganisms, viruses, proteins, enzymes, nucleic acids and low-molecular biological substances.

Examples of the specific cells to be detected include tumor cells circulating in the blood (circulating tumor cells) and other blood cells. Specific examples of the microorganisms and viruses include pathogenic microorganisms and pathogenic viruses that cause infectious diseases. Specific examples of the microorganisms and the viruses include mainly pathogens that cause the infectious diseases belonging to the first category to the fifth category, more specifically, human immunodeficiency viruses (HIV), hepatitis B viruses (HBV), hepatitis C viruses (HCV), severe acute respiratory syndrome (SARS) viruses, human papilloma viruses (HPV), influenza viruses, noroviruses and the like.

Examples of the proteins include: peptide hormones such as insulin and luteinizing hormone-releasing hormone agonist (LH-RH); tumor markers such as various immunoglobulins, albumin, carcinoembryonic antigen (CEA) and prostate-specific antigen (PSA); IgE as a marker for allergies such as pollinosis; thrombin as a marker for the coagulation fibrinolysis system; and C-reactive protein as a marker for inflammatory diseases.

Examples of the enzymes include aspartate aminotransferase (AST), glutamic pyruvic transaminase (GPT), lactate dehydrogenase (LDH), alkaline phosphatase and protein-tyrosine kinase.

Examples of the nucleic acids include nucleic acids derived from tumor cells that exist in the blood (circulating tumor DNA) and microRNA (ribonucleic acids).

Examples of the low-molecular biological substances include: blood components such as glucose, galactose, lactose, acetylcholine, glutamic acid, cholesterol, alcohol and 1,5-anhydroglucitol (1,5AG); and neurotransmitters such as dopamine, serotonin and norepinephrine.

As the substance (specific substance) that specifically interacts with the analyte, for example, the following can be used: an antibody; antibody fragment having antigen-recognition function; and an aptamer having antigen-recognition function. As the antibody, the following can be used: immunoglobulins such as immunoglobulin G (IgG) and immunoglobulin M (IgM). As the antibody fragment, Fab′ and Fab″ can be used. As the aptamer, a nucleic acid aptamer can be used. As the nucleic acid, DNA, polyamide nucleic acid and a modification thereof can be used, but not limited thereto.

In order to detect the analyte with a high sensitivity by the biosensor, it is necessary that the analyte should stably adsorb onto the single-walled carbon nanotube thin film 12 as the channel. It is possible to fix the analyte on the single-walled carbon nanotube thin film 12 via linker molecules having a particular structure. For this purpose, the antibody, the antibody fragment or the aptamer, which is a substance (specific substance) specifically interacting with the analyte, is combined with the linker molecules having affinity for the single-walled carbon nanotubes. When combining the antibody or the antibody fragment with the linker molecules, the succinimidyl group of the linker molecules are reacted with SH group or amino group that exists in the antibody or the antibody fragment.

It is preferable to use the linker molecule in which the polycyclic aromatic hydrocarbon structure is included because it adsorbs onto the surface of the single-walled carbon nanotube thin film 12 by a strong π electron interaction. Also, it is preferable to use the linker molecule having, in its structure, one or more substituents that can react with or adsorb onto or form a chelate with the specific substance that specifically interacts with the analyte, for example: reactive substituents such as an amino group, a carboxyl group, a hydroxyl group and a succinimide group; or hapten and/or chelate of biotin and biotin derivatives, digoxin and digoxin derivatives, digoxigenin and digoxigenin derivatives, fluorescein and fluorescein derivative, and theophylline.

Specific examples of the linker molecules include: acenaphthene derivatives; acetophenone derivatives; anthracene derivatives; diphenylacetylene derivatives; acridan derivatives; acridine derivatives; acridone derivatives; thioacridone derivatives; angelicin derivatives; anthracene derivatives; anthraquinone derivatives; azafluorene derivatives; azulene derivatives; benzil derivatives; carbazole derivatives; coronene derivatives; sumanene derivatives; biphenylene derivatives; fluorene derivatives; perylene derivatives; phenanthrene derivatives; phenanthroline derivatives; phenazine derivatives; benzophenone derivatives; pyrene derivatives; benzoquinone derivatives; biacetyl derivatives; bianthranil derivatives; fullerene derivatives; graphene derivatives; carotene derivatives; chlorophyll derivatives; chrysene derivatives; cinnoline derivatives; coumarin derivatives; curcumin derivatives; dansylamide derivatives; flavone derivatives; fluorenone derivatives; fluorescein derivatives; helicene derivatives; indenes derivatives; lumichrome derivatives; lumiflavin derivatives; oxadiazole derivatives; oxazole derivatives; periflanthene derivatives; perylene derivatives; phenanthrene derivatives; phenanthroline derivatives; phenazine derivatives; phenol derivatives; phenothiazine derivatives; phenoxazine derivatives; phthalazine derivatives; phthalocyanine derivatives; picene derivatives; porphyrin derivatives; porphycene derivatives; hemiporphycene derivatives; subphthalocyanine derivatives; psoralen derivatives; angelicin derivatives; purine derivatives; pyrene derivatives; pyrromethene derivatives; pyridyl ketone derivatives; phenyl ketone derivatives; pyrdyl ketone derivatives; thienyl ketone derivatives; furanyl ketone derivatives; quinazoline derivatives; quinoline derivatives; quinoxaline derivatives; retinal derivatives; retinol derivatives; rhodamine derivatives; riboflavin derivatives; rubrene derivatives; squarine derivatives; stilbene derivatives; tetracene derivatives; pentacene derivatives; anthraquinone derivatives; tetracenequinone derivatives; pentacenequinone derivatives; thiophosgene derivatives; indigo derivatives; thioindigo derivatives; thioxanthene derivatives; thymine derivatives; triphenylene derivatives; triphenylmethane derivatives; triaryl derivatives; tryptophan derivatives; uracil derivatives; xanthene derivative; ferrocene derivatives; azulene derivatives; biacetyl derivatives; terphenyl derivatives; terfuran derivatives; terthiophene derivatives; oligoaryl derivatives; fullerene derivatives; conjugated polyene derivatives; derivatives of a fused polycyclic aromatic compound containing a Group 14 element; and derivatives of a fused polycyclic heteroaromatic compound. However, the linker molecules are not limited thereto. Among others, pyrene derivatives such as 1-pyrenbutanoic acid succinimidyl ester are preferable.

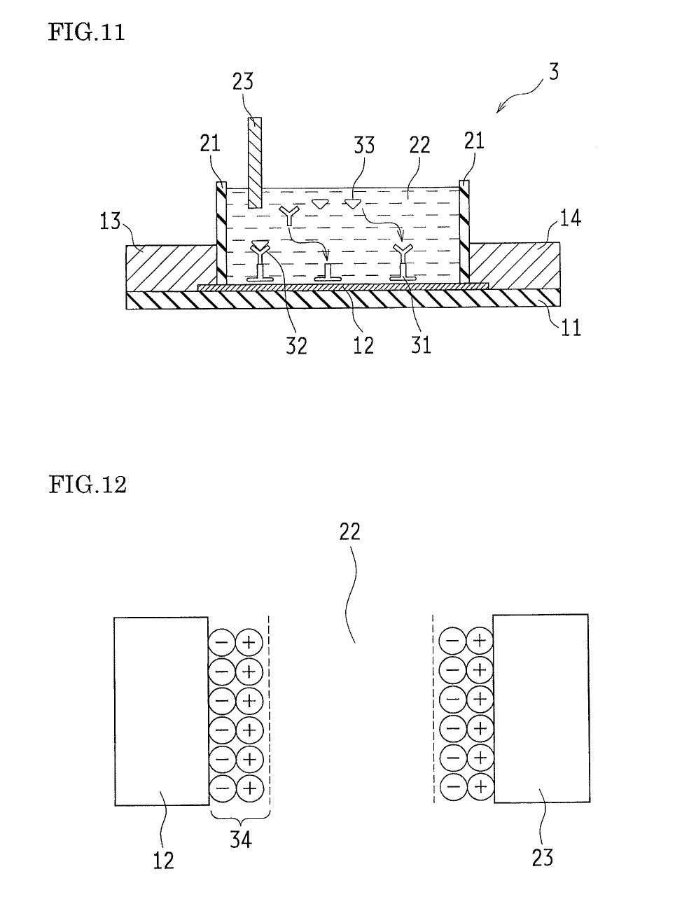

FIG. 11 is a schematic diagram showing a configuration of a biosensor 3 according to this embodiment. The biosensor 3 is configured, for example, by attaching the pool 21 made of silicone rubber on the field effect transistor 1 shown in FIGS. 1A and 1B, filling the pool 21 with the solution 22 containing an analyte, immersing the gate electrode 23 of the field effect transistor 1 in the solution 22, and connecting the bi-potentiostat 24 (not shown) to the respective electrodes 13, 14 and 23 of the field effect transistor 1. The solution 22 contains, for example, linker molecules 31, a specific substance 32 and an analyte 33. It is important that the part immersed in the solution 22 contain no noble metal impurity or the like.

The linker molecule 31 is the substance that has a high affinity for the single-walled carbon nanotube thin film 12 and has a functional group that combines with the specific substance 32. Also, the specific substance 32 is a specific substance that specifically interacts with the analyte 33. Thus, in the biosensor 3, the linker molecules 31 contained in the solution 22 adsorb onto the surface of the single-walled carbon nanotube thin film 12. The linker molecules 31 combine with the specific substance 32, and the specific substance 32 specifically interacts with the analyte 33. In this way, the analyte 33 stably adsorbs, selectively, onto the single-walled carbon nanotube thin film 12 as the channel.

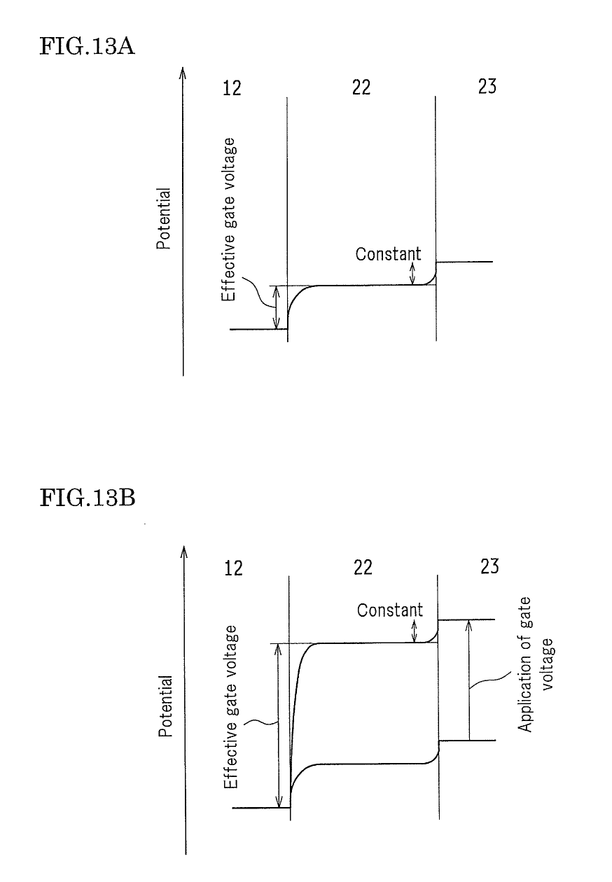

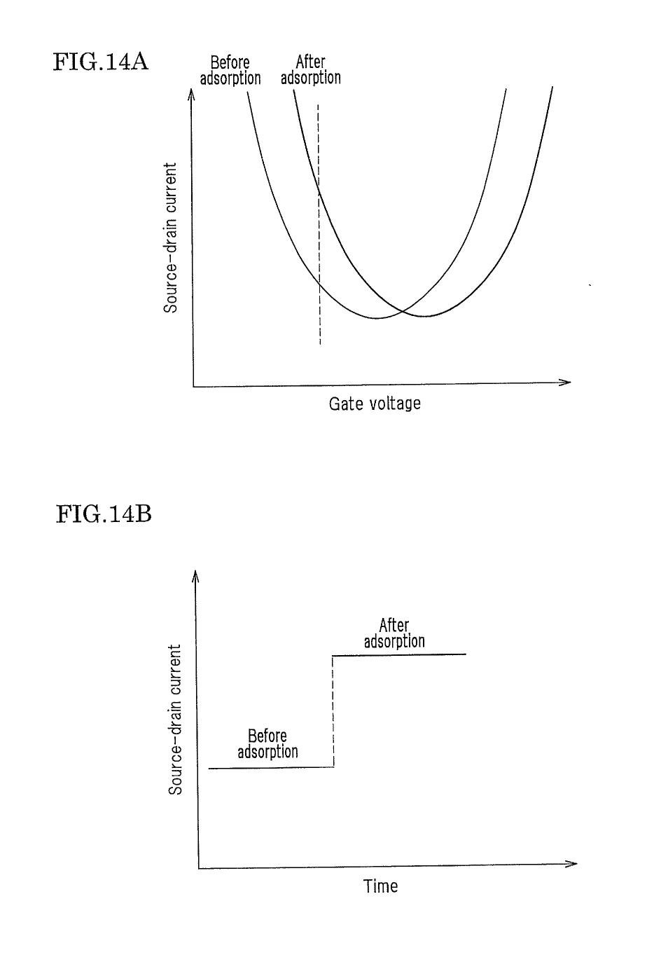

Next, description will be given on detection principle of the analyte 33 using the biosensor 3 of this embodiment with reference to FIGS. 12 to 14B. FIG. 12 is a pattern diagram showing formation of an electric double layer in the biosensor 3. FIGS. 13A and 13B are pattern diagrams each showing a change in a potential of the biosensor 3. FIGS. 14A and 14B are pattern diagrams each showing a change in a reference potential of the gate voltage and a change in a source-drain current.

As shown in FIG. 11, in the solution 22 of the biosensor 3, the analyte 33 adsorbs onto the single-walled carbon nanotube thin film 12 via the specific substance 32 and the linker molecules 31. Since the analyte 33 is proteins or ions having charges, such proteins or ions function as a capacitor in the vicinity of the surface of the single-walled carbon nanotube thin film 12 and the surface of the gate electrode 23 as shown in FIG. 12. Thus, the electric double layer is formed.

FIGS. 13A and 13B are pattern diagrams schematically showing each potential of the single-walled carbon nanotube thin film 12, the solution 22 and the gate electrode 23. FIG. 13A shows the potential before the analyte 33 adsorbs onto the surface of the single-walled carbon nanotube thin film 12. FIG. 13B shows the potential after the analyte 33 adsorbs onto the surface of the single-walled carbon nanotube thin film 12. In the vicinity of the interface between the single-walled carbon nanotube thin film 12 and the solution 22, the effective gate voltage is affected, after the adsorption, by the electric double layer, so that the effective gate voltage changes and increases.

This means that, as shown in FIG. 14A, the reference potential of the gate voltage changes after the adsorption of the analyte 33, which leads to a change in the relation between the gate voltage and the source-drain current. Therefore, even when the constant gate voltage, which is indicated by the dashed line in FIG. 14A, is applied to the gate electrode 23, the detected value of the source-drain current changes before and after the adsorption. FIG. 14B is a graph showing the change in the source-drain current before and after the adsorption of the analyte 33 with the detected value of the source-drain current as ordinate and the time as abscissa.

That is, when the analyte 33 adsorbs onto the surface of the single-walled carbon nanotube thin film 12, the electric double layer is formed. Accordingly, the gate voltage is effectively applied, which leads to the change in the detected value of the source-drain current. Therefore, by measuring the change in the source-drain current of the biosensor 3, it is possible to detect ions or proteins as the analyte 33.

Also, in this embodiment, the length of the specific substance 32 that adsorbs onto the single-walled carbon nanotube thin film 12, or the total length of the linker molecule 31 and the specific substance 32 should be shorter than the length corresponding to the Debye length of the electric double layer. Normally, the total length of the linker molecule 31 and the specific substance 32 is 100 nm or less, preferably 10 nm or less, more preferably 5 nm or less, in particular 3 nm or less.

In the present invention, the analyte 33 adsorbs onto the single-walled carbon nanotube thin film 12 of the sensor 2 or the biosensor 3, thus the field effect transistor 1 as the transducer converts an electric displacement into a detectable signal. In consideration of the sensitivity of the sensor 2 or the biosensor 3, the carrier mobility of the field effect transistor 1 is preferably high, and normally it is 0.1 cm²/V·s or more, preferably 1 cm²/V·s or more, more preferably 100 cm²/V·s or more. Also, the field effect transistor 1 can be arranged in array by the conventional microfabrication technology, thus, it is easy to form a multiplex sensor 2 or biosensor 3 by integration.

Furthermore, the sensor 2 or the biosensor 3 detects only specific adsorption in the electric double layer formed on the surface as the electric signal. Therefore, it is not affected by noise due to nonspecific adsorption that is likely to occur outside of the electric double layer, and a high sensitivity of the sensor can be expected. Thus, the sensor 2 or the biosensor 3 in which the field effect transistor 1 of the present invention is used as the transducer can selectively detect the very small amount of analyte 33 as the target with a high sensitivity.

### [Example 10] Detection of BSA

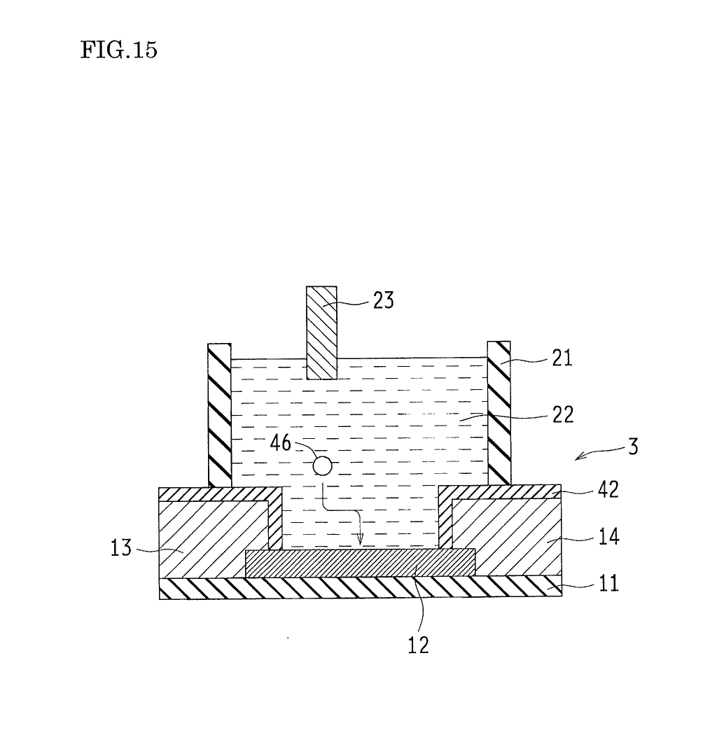

FIG. 15 is a pattern diagram showing a BSA detection method by the biosensor 3. In the BSA detection, the pH value of the solution 22 was maintained constant using, as the buffer solution, a phosphate buffer solution (pH 6.8) (manufactured by HORIBA, Ltd.), the concentration of which was adjusted to 10 mM by being diluted with the ultrapure water. Then, BSA 46 (trade name “A0281-250 mg”, manufactured by Sigma-Aldrich Co., LLC.), which was a protein as the analyte, was prepared as a solution, the concentration of which was adjusted using the phosphate buffer solution (pH 6.8) as the solvent. The BSA concentration in the solution 22 was changed by dripping the thus prepared BSA 46, and the change in the source-drain current I_{sd} with respect to time was recorded.

The measurement was conducted by applying the gate voltage (in this case, top gate voltage) V_{g}=−0.3 V and the source-drain voltage V_{sd}=0.1 V. In the measurement procedure, first, the pool 21 made by cutting out silicone rubber was disposed on the substrate 11 so as to serve as a partition wall for keeping the solution on the substrate. Then, the pool 21 was well rinsed by the phosphate buffer solution (pH 6.8). After that, 50 μl phosphate buffer solution (pH 6.8) was dripped into the pool 21. The recording of the change in the source-drain current was started, and 150 seconds later, 50 μl BSA, whose concentration was adjusted to 10 nM using the phosphate buffer solution (pH 6.8) as the solvent, was dripped and sufficiently stirred. After that, the solution was adjusted to 50 μl. Another 180 seconds later, 50 μl BSA, whose concentration was adjusted to 100 nM using the phosphate buffer solution (pH 6.8) as the solvent, was dripped and sufficiently stirred. After that, the solution was adjusted to 50 μl. Another 180 seconds later, 50 μl BSA, whose concentration was adjusted to 1000 nM using the phosphate buffer solution (pH 6.8) as the solvent, was dripped and sufficiently stirred. The change in the source-drain current value I_{sd} was recorded for further 180 seconds.

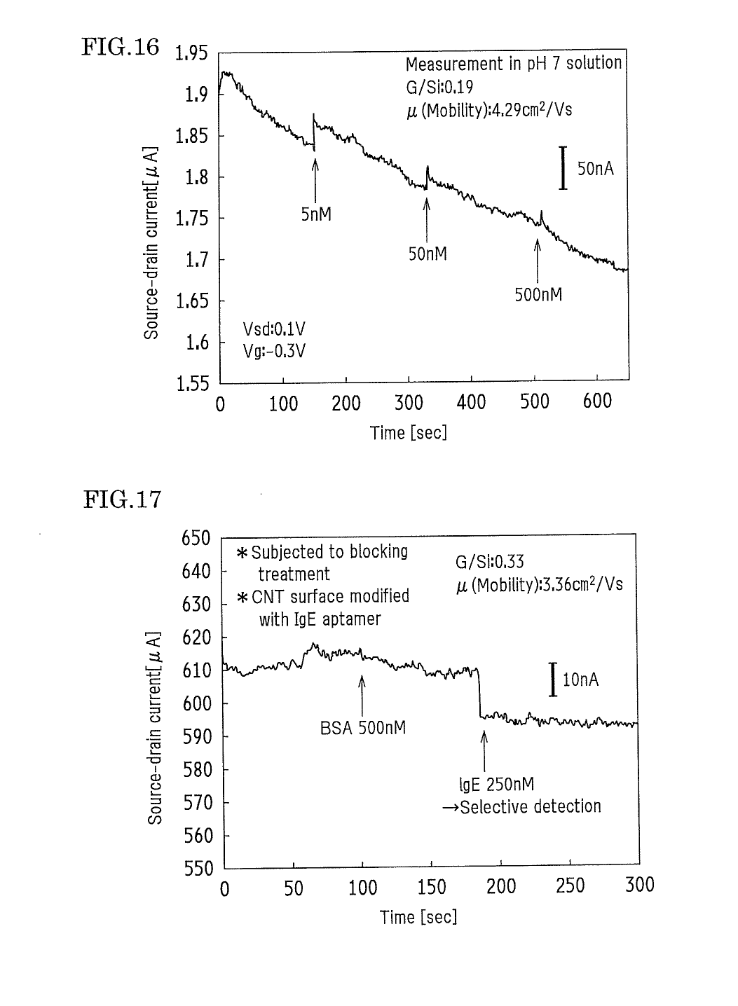

FIG. 16 is a graph showing measurement results of the BSA concentration in the electrolyte solution as the solution 22 using the biosensor 3 shown in FIG. 15. As can be seen from FIG. 16, when the BSA concentration of the solution 22 is sequentially changed from 0 M to 5 nM, 50 nM and 500 nM, it is observed that the source-drain current value changes in a stepwise manner during dripping the BSA solution. This change means that an effect by effectively applied negative gate voltage, which is caused by adsorption of the BSA having negative charges in the pH 6.8 solution onto the surface of the single-walled carbon nanotube thin film 12, was detected as the change in the source-drain current value.

### [Example 11] Detection of IgE

FIG. 17 is a graph showing results of IgE selectively detected in the electrolyte solution as the solution 22 using the biosensor 3 shown in FIG. 11. In the IgE selective detection, the pH value of the solution 22 was maintained constant using, as the buffer solution, a phosphate buffer solution (pH 6.8) (manufactured by HORIBA, Ltd.), the concentration of which was adjusted to 10 mM by being diluted with the ultrapure water. Then, IgE (trade name “HUMAN IgE”, manufactured by YAMASA CORPORATION), which was a protein as the analyte 33, was prepared as a solution, the concentration of which was adjusted using the phosphate buffer solution (pH 6.8) as the solvent. The IgE concentration in the solution 22 was changed by dripping the thus prepared IgE, and the change in the source-drain current I_{sd} with respect to time was recorded.

The measurement was conducted by applying the gate voltage (in this case, top gate voltage) V_{g}=−0.3 V and the source-drain voltage V_{sd}=0.1 V. In the measurement procedure, first, the pool 21 made by cutting out silicone rubber was disposed on the substrate 11 so as to serve as a partition wall for keeping the solution on the substrate. Then, the pool 21 was filled with 1-pyrenbutanoic acid succinimidyl ester (trade name “P-130”, manufactured by LIFE Technologies Corp.) as the linker molecules 31, whose concentration was adjusted to 1 mM using methanol as the solvent. One hour later, the solution in the pool 21 was removed and the pool 21 was well rinsed by methanol, and furthermore well rinsed by the phosphate buffer solution (pH 6.8). Then, the pool 21 was filled with an IgE aptamer having antigen-recognition function (manufactured by FASMAC Co., Ltd.) as the specific substance 32, whose concentration was adjusted to 100 nM using the phosphate buffer solution (pH 6.8) as the solvent.

12 hours later, the solution in the pool 21 was removed and the pool 21 was well rinsed by the phosphate buffer solution (pH 6.8). After that, 50 μl phosphate buffer solution (pH 6.8) was dripped into the pool 21. For blocking treatment, 50 μl BSA 46 (trade name “A0281-250 mg”, manufactured by Sigma-Aldrich Co., LLC.), whose concentration was adjusted to 50 μM using the phosphate buffer solution (pH 6.8) as the solvent, was dripped and sufficiently stirred. After that, the solution was adjusted to 50 μl. The recording of the source-drain current was started, and 100 seconds later, 50 μl BSA, whose concentration was adjusted to 1000 nM using the phosphate buffer solution (pH 6.8) as the solvent, was dripped and sufficiently stirred. After that, the solution was adjusted to 50 μl. Another 90 seconds later, 50 μl IgE, whose concentration was adjusted to 500 nM using the phosphate buffer solution (pH 6.8) as the solvent, was dripped and sufficiently stirred. The change in the source-drain current value I_{sd} was recorded for further 110 seconds.

As can be seen from FIG. 17, no change in the current value is observed during dripping the BSA solution. On the other hand, the change in the current value is observed during dripping the IgE solution. This means that no change in the current value is observed because the BSA solution does not adsorb onto the surface of the single-walled carbon nanotubes after blocking treatment, and that an effect by effectively applied positive gate voltage is detected as the change in the source-drain current value because the IgE having positive charges in the pH 6.8 solution selectively adsorbs onto the IgE aptamer. It can be seen that the IgE can be selectively detected by modifying the surface of the single-walled carbon nanotubes with the IgE aptamer and furthermore by subjecting to blocking treatment.

### [Example 12] Detection of IgE

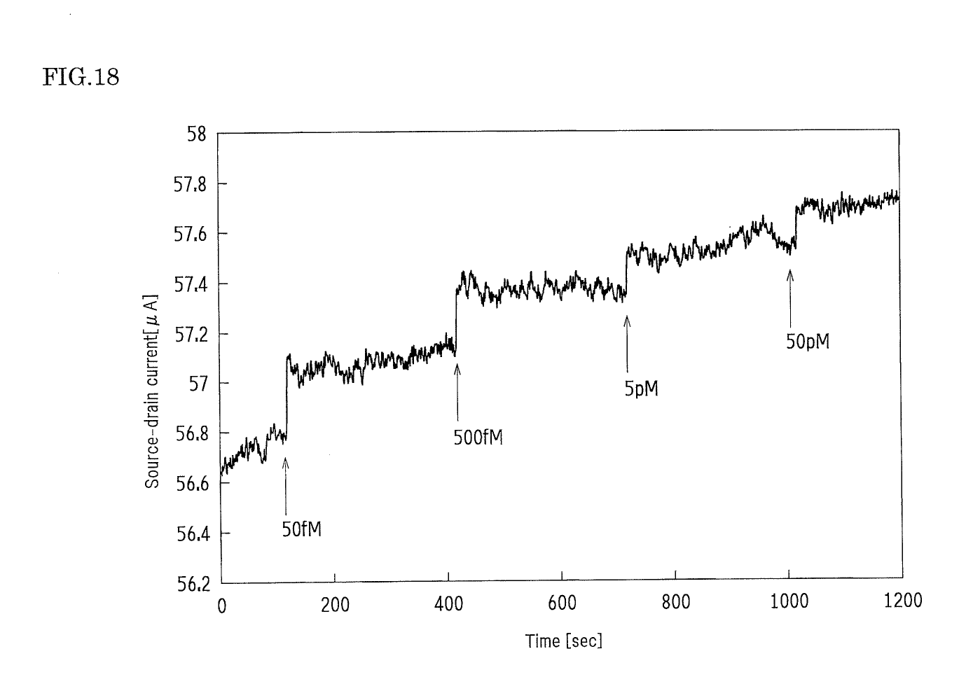

FIG. 18 is a graph showing detection results of IgE in the electrolyte solution as the solution 22 in a manner similar to that in Example 11, using the biosensor 3 shown in FIG. 11. In the IgE selective detection, the pH value of the solution 22 was maintained constant using, as the buffer solution, a phosphate buffer solution (pH 6.8) (manufactured by HORIBA, Ltd.), the concentration of which was adjusted to 10 mM by being diluted with the ultrapure water. Then, IgE (trade name “HUMAN IgE”, manufactured by YAMASA CORPORATION), which was a protein as the analyte 33, was prepared as a solution, the concentration of which was adjusted using the phosphate buffer solution (pH 6.8) as the solvent. The IgE concentration in the solution 22 was changed by dripping the prepared IgE, and the change in the source-drain current La with respect to time was recorded.

The measurement was conducted by applying the gate voltage (in this case, top gate voltage) V_{g}=−0.3 V and the source-drain voltage V_{sd}=0.1 V. In the measurement procedure, first, the pool 21 made by cutting out silicone rubber was disposed on the substrate 11 so as to serve as a partition wall for keeping the solution on the substrate. Then, the pool 21 was filled with 1-pyrenbutanoic acid succinimidyl ester (trade name “P-130”, manufactured by LIFE Technologies Corp.) as the linker molecules 31, whose concentration was adjusted to 1 mM using methanol as the solvent. One hour later, the solution in the pool 21 was removed and the pool 21 was well rinsed by methanol, and furthermore well rinsed by the phosphate buffer solution (pH 6.8). Then, the pool 21 was filled with an IgE aptamer having antigen-recognition function (manufactured by FASMAC Co., Ltd.) as the specific substance 32, whose concentration was adjusted to 100 nM using the phosphate buffer solution (pH 6.8) as the solvent.

12 hours later, the solution in the pool 21 was removed and the pool 21 was well rinsed by the phosphate buffer solution (pH 6.8). After that, 50 μl phosphate buffer solution (pH 6.8) was dripped into the pool 21. For blocking treatment, 50 μl BSA 46 (trade name “A0281-250 mg”, manufactured by Sigma-Aldrich Co., LLC.), whose concentration was adjusted to 50 μM using the phosphate buffer solution (pH 6.8) as the solvent, was dripped and sufficiently stirred. After that, the solution was adjusted to 50 μl. The recording of the source-drain current was started, and 120 seconds later, 50 μl IgE, whose concentration was adjusted to 100 fM using the phosphate buffer solution (pH 6.8) as the solvent, was dripped and sufficiently stirred. After that, the solution was adjusted to 50 μl. Another 300 seconds later, 50 μl IgE, whose concentration was adjusted to 1 pM using the phosphate buffer solution (pH 6.8) as the solvent, was dripped and sufficiently stirred. After that, the solution was adjusted to 50 μl. Another 300 seconds later, 50 μl IgE, whose concentration was adjusted to 10 pM using the phosphate buffer solution (pH 6.8) as the solvent, was dripped and sufficiently stirred. After that, the solution was adjusted to 50 μl. Another 300 seconds later, 50 μl IgE, whose concentration was adjusted to 100 pM using the phosphate buffer solution (pH 6.8) as the solvent, was dripped and sufficiently stirred. The change in the source-drain current value I_{sd} was recorded for further 180 seconds.

In FIG. 18, the changes in the current value indicated by the arrows show, respectively, the time points at which 50 fM IgE, 500 fM IgE, 5 pM IgE and 50 pM IgE were detected. As can be seen from FIG. 18, not only the IgE can be selectively detected, but also the low-concentration IgE (such as 50 fM) can be detected.

### Fourth Embodiment

Next, as the fourth embodiment of the present invention, description will be given on another aspect of the single-walled carbon nanotube thin film 12 of the field effect transistor 1 according to the first embodiment. In this embodiment, the channel constituted by the single-walled carbon nanotube thin film 12 is patterned and slits are formed between the source electrode 13 and the drain electrode 14, so that the channel has a strip structure. Since only the pattern shape of the single-walled carbon nanotube thin film 12 is different from that of the first embodiment, the redundant description is omitted.

### Example 13

### (a) Preparation of (Long) Single-Walled Carbon Nanotube Thin Film

On a 1 cm square thermally oxidized silicon substrate as the substrate 11, which was made by forming the silicon oxide layers 11b respectively on the surface and the rear surface of the silicon substrate 11a by oxidation, ethanol dispersion liquid containing 2.0 mass % hydrogenated nanodiamond (trade name: “Ustalla (registered trade mark) Type C”, manufactured by Nippon Kayaku Co., Ltd.) having the particle size distribution of 5 nm to 15 nm and the metallic impurities of 100 mass ppm was applied by the spin coating method so as to obtain a substrate on which the hydrogenated nanodiamond was applied. The spin coating conditions were the same as those in Example 1. Sequentially, the substrate on which the hydrogenated nanodiamond had been applied was placed in the heating furnace so as to be subjected to the heating treatment in the atmosphere at 600° C. for 15 minutes, thus the hydrogenated nanodiamond growth nuclei having the particle size of 0.5 nm to 4 nm were obtained. The substrate was placed in the CVD reactor handling the multiple temperature conditions as shown in FIG. 3 and the single-walled carbon nanotube thin film 12 was obtained by flowing, for 2 minutes, gas composed of 10 sccm acetylene (diluted with argon as dilution gas to 2 vol. %) as the growth gas and 10 sccm argon-hydrogen carrier gas, and sequentially by flowing, for 58 minutes, gas composed of 2 sccm acetylene (diluted with argon as dilution gas to 2 vol. %) and 18 sccm argon-hydrogen carrier gas, under the condition that the temperature on the upstream side was 850° C., the temperature in the vicinity of the substrate was 780° C. and the pressure was 500 Pa.

When the obtained single-walled carbon nanotube thin film was evaluated using the Raman spectrometer, the ratio I(G)/I(Si) was 0.11, and the ratio I(G)/I(D) was 3.2. Thus, it was confirmed that the obtained single-walled carbon nanotube thin film had the density lower than that obtained in Example 1(a). Using the obtained single-walled carbon nanotube thin film, a field effect transistor was manufactured in the same way as Example 1.

### Example 14

### (a) Preparation of (Short) Single-Walled Carbon Nanotube Thin Film

On a 1 cm square thermally oxidized silicon substrate as the substrate 11, which was made by forming the silicon oxide layers 11b respectively on the surface and the rear surface of the silicon substrate 11a by oxidation, ethanol dispersion liquid containing 2.0 mass % hydrogenated nanodiamond (trade name: “Ustalla (registered trade mark) Type C”, manufactured by Nippon Kayaku Co., Ltd.) having the particle size distribution of 5 nm to 15 nm and the metallic impurities of 100 mass ppm was applied by the spin coating method so as to obtain a substrate on which the hydrogenated nanodiamond was applied. The spin coating conditions were the same as those in Example 1. Sequentially, the substrate on which the hydrogenated nanodiamond had been applied was placed in the heating furnace so as to be subjected to the heating treatment in the atmosphere at 600° C. for 15 minutes, thus the hydrogenated nanodiamond growth nuclei having the particle size of 0.5 nm to 4 nm were obtained. The substrate was placed in the CVD reactor handling the multiple temperature conditions as shown in FIG. 3 and the single-walled carbon nanotube thin film 12 was obtained by flowing, for 30 minutes, gas composed of 10 sccm acetylene (diluted with argon as dilution gas to 2 vol. %) as the growth gas and 10 sccm argon-hydrogen carrier gas, under the condition that the temperature on the upstream side was 850° C., the temperature in the vicinity of the substrate was 780° C. and the pressure was 500 Pa.

When the obtained single-walled carbon nanotube thin film was evaluated using the Raman spectrometer, the ratio I(G)/I(Si) was 0.14, and the ratio I(G)/I(D) was 2.7. Thus, it was confirmed that the obtained single-walled carbon nanotube thin film had the density lower than that obtained in Example 1(a). Using the obtained single-walled carbon nanotube thin film, a field effect transistor was manufactured in the same way as Example 1.

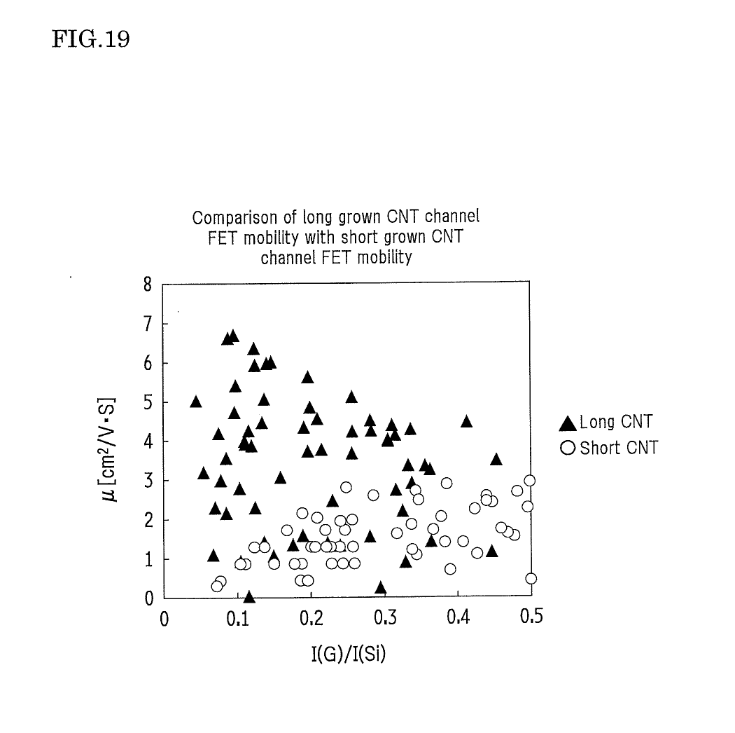

FIG. 19 is a graph plotted with each measured ratio I(G)/I(Si) as abscissa and each carrier mobility μ as ordinate regarding the field effect transistors 1 of Examples 1 and 2, in which the single-walled carbon nanotubes constituting the single-walled carbon nanotube thin film 12 are classified into the long carbon nanotubes and the short carbon nanotubes. In this graph, the long single-walled carbon nanotubes are plotted with black triangles while the short single-walled carbon nanotubes are plotted with outlined circles. As can clearly be seen from FIG. 19, there is a tendency that the carrier mobility μ increases when the single-walled carbon nanotube thin film is composed of the long single-walled carbon nanotubes regardless of the ratio I(G)/I(Si).

Generally, when a single carbon nanotube constitutes the channel, the obtained field effect transistor reflects the properties of the carbon nanotube. However, when a plurality of single-walled carbon nanotubes constitutes the single-walled carbon nanotube thin film 12, the carrier mobility μ is suppressed due to number of contacts among the single-walled carbon nanotubes. Since the long single-walled carbon nanotubes have a small number of contacts among the single-walled carbon nanotubes and thus have a large carrier mobility μ compared with the short single-walled carbon nanotubes, it is preferable to prepare the long single-walled carbon nanotubes when forming the single-walled carbon nanotube thin film 12 of the field effect transistor 1.

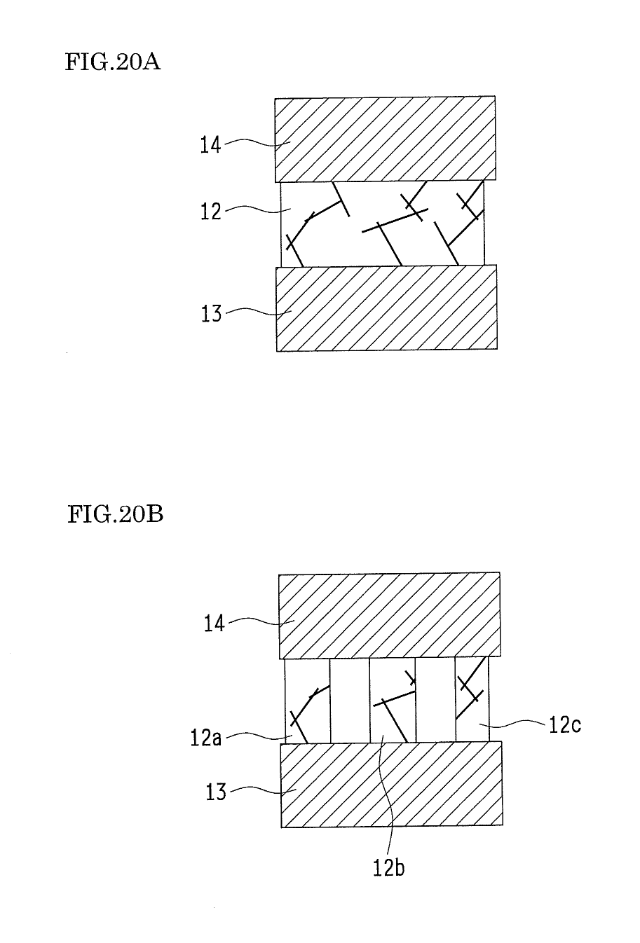

However, when growing a plurality of single-walled carbon nanotubes, the single-walled carbon nanotubes having metallic properties are mixed with the single-walled carbon nanotubes having semiconductor properties. FIG. 20A is a pattern diagram showing a case in which the single-walled carbon nanotubes having metallic properties are mixed. In FIG. 20A, the single-walled carbon nanotubes having semiconductor properties are omitted.

When a plurality of single-walled carbon nanotubes having metallic properties is mixed in the single-walled carbon nanotube thin film 12 constituting the channel, the single-walled carbon nanotubes having metallic properties make contact with one another as shown in the ellipse in FIG. 20A, thus a crosslinking structure is formed by the single-walled carbon nanotubes having metallic properties between the source electrode 13 and the drain electrode 14.

In such a crosslinking structure, channel paths are formed by only the single-walled carbon nanotubes having metallic properties. Thus, a leakage current is generated, which results in increase in the off current. When the leakage current is generated, the properties of the field effect transistor 1 are deteriorated. Accordingly, the sensitivity of the sensor 2 or the biosensor 3 using the field effect transistor 1 is also deteriorated. In particular, when the long single-walled carbon nanotubes are used for the purpose of improving the carrier mobility, the above crosslinking structure is highly likely to be formed.

In this embodiment, as shown in FIG. 20B, slits are formed when patterning, thus the single-walled carbon nanotube thin film 12 having a strip structure including a plurality of regions 12a, 12b and 12c. The widths of the respective regions 12a, 12b and 12c are normally in the range of 1 μm to 100 μm, preferably in the range of about 1 μm to 10 μm. In this way, when the slits are formed in the single-walled carbon nanotube thin film 12, the crosslinking structure of the single-walled carbon nanotubes having metallic properties is cut. Thus, it is possible to reduce the above-described channel paths so as to decrease the leakage current. Accordingly, the properties of the field effect transistor 1 can be prevented from being deteriorated, which enables to maintain the sensitivity of the sensor 2 or the biosensor 3 using the field effect transistor 1. The shape or number of the slits may be appropriately changed.

The channel current of the field effect transistor 1 is expressed by (surface density of charge)×(electric field)×(channel width)×(carrier mobility). In the single-walled carbon nanotube thin film 12 having the strip structure as shown in FIG. 20B, both of the channel width and the channel current are decreased compared with the structure in FIG. 20A. These changes mean the deterioration of the properties of the field effect transistor 1 or the deterioration of the sensitivity of the sensor 2 of the biosensor 3 using the field effect transistor 1.

However, the single-walled carbon nanotube thin film 12 of the present invention is made by growing the single-walled carbon nanotubes, by the chemical vapor deposition method, using particles of a nonmetallic material (nanodiamond) as the growth nuclei, the nonmetallic material containing 500 mass ppm or less metallic impurities that contain a metal and its compounds. Accordingly, as exemplarily shown in FIG. 7, it has a large carrier mobility. Therefore, even when the channel width is decreased due to the strip structure of the single-walled carbon nanotube thin film 12, improvement of the carrier mobility can sufficiently compensate for decrease in the channel current. That is, the field effect transistor 1 having a strip structure by forming slits, and the sensor 2 or the biosensor 3 using such a field effect transistor 1, can prevent the leakage current due to the crosslinking structure formed by the single-walled carbon nanotubes having metallic properties, while compensating for the decrease in the channel current caused by the decrease in the channel width. Thus, it is possible to maintain good properties.

### Fifth Embodiment

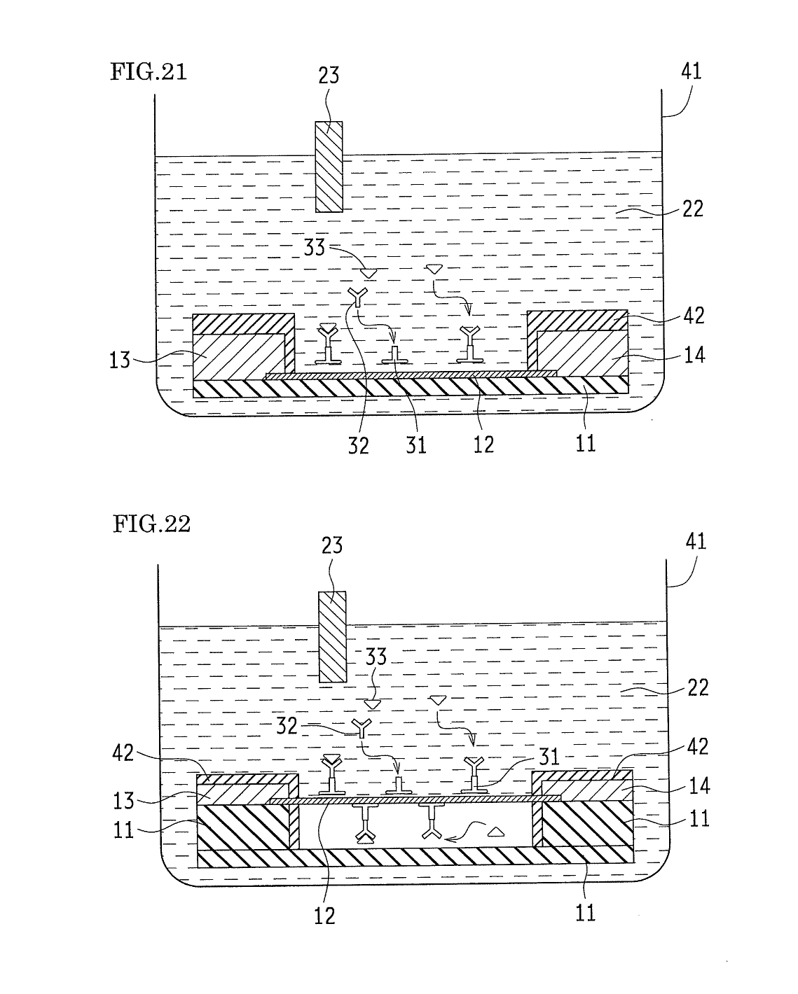

Next, as the fifth embodiment of the present invention, a variation of the biosensor 3 shown in the third embodiment will be described with reference to FIG. 21. The description that overlaps with the third embodiment is omitted. In this embodiment, as shown in FIG. 21, the biosensor 3 is configured by filling a container 41 with the solution 22, and immersing the field effect transistor 1 in the container 41. Also, the coating layer 42 is formed on each surface of the single-walled carbon nanotube thin film 12, the source electrode 13 and the drain electrode 14 of the field effect transistor 1. Here, in place of the container 41, the pool 21 may be used similarly to the third embodiment.

In this embodiment, by forming the coating layer 42, the oxidation reaction and the reduction reaction accompanying voltage application to the biosensor 3 are prevented from occurring in the single-walled carbon nanotube thin film 12, the source electrode 13 and the drain electrode 14. Thus, it is possible to suppress deterioration of the sensing sensitivity. Any material can be used as the coating layer 42 provided that such a material can prevent the oxidation reaction and the reduction reaction in the respective parts of the biosensor 3. For example, an alumina layer or a silicon oxide film are used as the coating layer 42.

### Sixth Embodiment

Next, as the sixth embodiment of the present invention, another aspect of the biosensor 3 shown in the third embodiment will be described with reference to FIG. 22. The description that overlaps with the third embodiment is omitted. In this embodiment, as shown in FIG. 22, the single-walled carbon nanotube thin film 12, which constitutes the channel of the field effect transistor 1, is disposed under the electrodes between the source electrode 13 and the drain electrode 14, and the single-walled carbon nanotube thin film 12 is separated from the surface of the substrate 11 by partially etching the substrate 11. Thus, the single-walled carbon nanotube thin film 12 is configured to be free-standing. Also, on each surface of the source electrode 13 and the drain electrode 14, the coating layer 42 may be formed.

Examples of the methods in which the single-walled carbon nanotube thin film 12 is disposed separated from the substrate 11 include a method in which the single-walled carbon nanotube thin film 12 is grown, in a manner similar to that in the first embodiment, on a separately prepared substrate for growth, and then the grown single-walled carbon nanotube thin film 12 is transferred onto the substrate 11 on which the source electrode 13 and the drain electrode 14 are formed.

In this embodiment, since the single-walled carbon nanotube thin film 12 as the channel is separated from the substrate 11, scattering of the channel current is suppressed, thus the change in the amount of the current can be detected with a high sensitivity.

### Seventh Embodiment

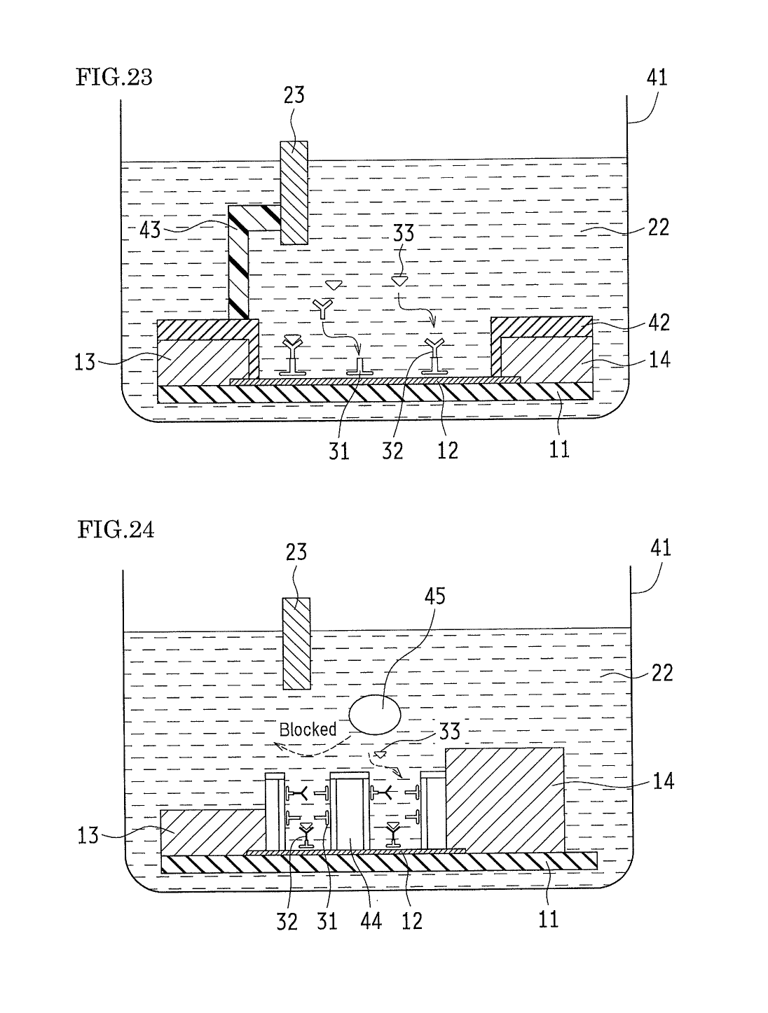

Next, as the seventh embodiment of the present invention, another aspect of the biosensor 3 shown in the third embodiment will be described with reference to FIG. 23. The description that overlaps with the third embodiment is omitted. In this embodiment, as shown in FIG. 23, the gate electrode 23 is fixed to the field effect transistor 1 using a support member 43 made of an insulating material. The position where the support member 43 is disposed is not particularly limited. The support member 43 can be disposed in any region provided that it does not affect the single-walled carbon nanotube thin film 12, the source electrode 13 and the drain electrode 14.

In this embodiment, since the gate electrode 23 is fixed using the support member 43, the gate electrode 23 can be integrated into the biosensor 3. Thus, it becomes easy to handle the device.

### Eighth Embodiment

Next, as the eighth embodiment of the present invention, another aspect of the biosensor 3 shown in the third embodiment will be described with reference to FIG. 24. The description that overlaps with the third embodiment is omitted. In this embodiment, as shown in FIG. 24, a porous structure 44 is disposed on the single-walled carbon nanotube thin film 12. The linker molecules 31 are respectively positioned in holes of the porous structure 44. Examples of the porous structures 44 include porous alumina having a nanohole structure.

In this embodiment, since the porous structure 44 is disposed on the single-walled carbon nanotube thin film 12, it is possible to extend the channel area, which results in improvement of the sensitivity of the biosensor 3. Also, it is preferable that the size of the nanoholes formed in the porous structure 44 are smaller than the size of the normal nonspecific adsorption proteins (e.g., bovine serum albumin (BSA)) and furthermore larger than the size of the proteins of the analyte 33. In this way, nonspecific adsorption proteins 45 other than the analyte 33 do not enter the nanoholes, thus it is possible to select more efficiently the analyte 33 and make it adsorb, which results in improvement of the detection sensitivity of the biosensor 3.

### Ninth Embodiment

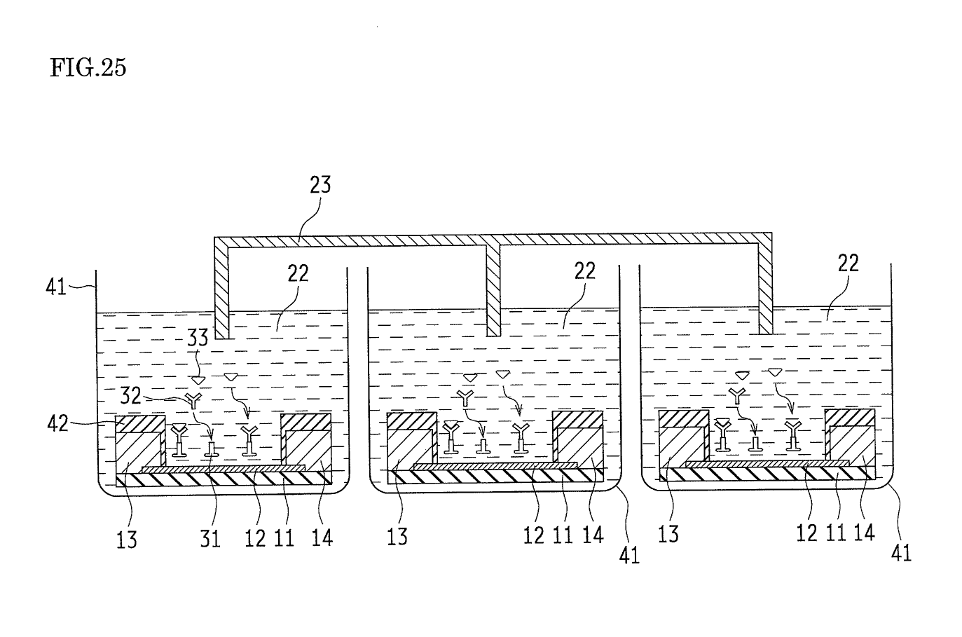

Next, as the ninth embodiment of the present invention, another aspect of the biosensor 3 shown in the third embodiment will be described with reference to FIG. 25. The description that overlaps with the third embodiment is omitted. In this embodiment, as shown in FIG. 25, a plurality of the biosensors 3, each immersed in the corresponding container 41, is disposed in parallel. Each gate electrode 23 of the corresponding biosensor 3 is connected in parallel. Also, in each biosensor 3, the kind of the specific substance 32 that modifies the surface of the corresponding single-walled carbon nanotube thin film 12 is different from each other.

In this embodiment, since the plurality of the biosensors 3 is disposed in parallel and furthermore the kind of the specific substance 32 for each of the biosensor 3 is different from each other, it is possible to detect a plural kinds of proteins according to the specific substance 32.

### Tenth Embodiment

Next, as the tenth embodiment of the present invention, another aspect of the biosensor 3 shown in the third embodiment will be described. The description that overlaps with the third embodiment is omitted. In the third embodiment to the ninth embodiment as described above, in order to perform detection by the biosensor 3, the pool 21 or the container 41 is filled with the solution 22 containing the analyte 33. In this embodiment, the solution 22 is dripped on the channel of the field effect transistor 1, and after that the solvent of the solution 22 is evaporated. In order to evaporate the solvent, various methods can be used, including dry gas jetting and heating.

The amount of the solvent to be evaporated can be appropriately set, however, the solution 22 should remain to the extent that the gate electrode 23 makes contact with the solution 22. Since the solvent of the solution 22 is evaporated, it is possible to enhance the probability that the analyte 33 adsorbs onto the single-walled carbon nanotube thin film 12 as well as to remove noise derived from the solvent.

The field effect transistor of the present invention includes a source electrode, a drain electrode, a channel formed between the source electrode and the drain electrode, and a gate electrode. The channel is constituted by a single-walled carbon nanotube thin film that is grown, by the chemical vapor deposition method, using particles of a nonmetallic material as growth nuclei, the nonmetallic material containing 500 mass ppm or less metallic impurities that contain a metal and its compounds.

In the field effect transistor of the present invention, the channel is constituted by the single-walled carbon nanotube thin film that is grown, by the chemical vapor deposition method, using the particles of the nonmetallic material as the growth nuclei, the nonmetallic material containing 500 mass ppm or less metallic impurities that contain a metal and its compounds. Thus, the carrier mobility is improved and good transistor properties can be obtained. Also, it is possible to manufacture the field effect transistors having uniform properties by simple steps at low costs.

Also, in the field effect transistor of the present invention, it is preferable to use nanodiamond as the growth nuclei.

Like this, using nanodiamond as the growth nuclei, it is possible to further reduce the metallic impurities contained in the growth nuclei.

Also, in the sensor of the present invention, the field effect transistor of the present invention is used as a transducer.

Like this, the sensor of the present invention includes, as the transducer, the field effect transistor in which the channel is constituted by the single-walled carbon nanotube thin film that is grown, by the chemical vapor deposition method, using the particles of the nonmetallic material as the growth nuclei, the nonmetallic material containing 500 mass ppm or less metallic impurities that contain a metal and its compounds. Thus, it is possible to detect the very small amount of analyte with a high sensitivity. Furthermore, the field effect transistor can be arranged in array by the conventional microfabrication technology, thus, it is easy to form a multiplex sensor by integration.

Also, in the sensor of the present invention, the channel may be modified with a specific substance that specifically interacts with the analyte.

In this way, by modifying the channel with the substance (specific substance) that specifically interacts with the analyte, it is possible to selectively detect the analyte with a high sensitivity.

Also, in the sensor of the present invention, the specific substance that specifically interacts with the analyte may be an antibody, antibody fragment or an aptamer.

Thus, by using the antibody, the antibody fragment or the aptamer as the substance (specific substance) that specifically interacts with the analyte, it is possible to suitably select the specific substance depending on the kind of the analyte, which leads to selective detection of the analyte with a high sensitivity.

Also, in the sensor of the present invention, the specific substance, which specifically interacts with the analyte, may be fixed onto the single-walled carbon nanotube thin film via linker molecules.

Like this, by fixing the specific substance via the linker molecules, the specific substance and the analyte can effectively adsorb onto the surface of the channel via the linker molecules that have a high affinity for the single-walled carbon nanotube thin film and that combine with the specific substance.

Also, in the sensor of the present invention, the analyte may be cells, microorganisms, viruses, proteins, enzymes, nucleic acids or low-molecular biological substances.

The present invention may be embodied in other forms without departing from the gist or essential characteristics thereof. The foregoing examples are therefore to be considered in all respects as illustrative and not limiting. The scope of the present invention is indicated by the appended claims rather than by the foregoing description, and all modifications and changes that come within the meaning and range of equivalency of the claims are intended to be embraced therein. Also, this application claims priority on Patent Application No. 2014-162737 filed in Japan on Aug. 8, 2014. All of the publications, patents and patent applications (including the above Japanese patent application) cited herein are hereby incorporated by reference in their entirety.

### REFERENCE SIGNS LIST

1 Field effect transistor
2 Sensor
3 Biosensor
11 Substrate
12 Single-walled carbon nanotube thin film
13 Source electrode
14 Drain electrode
21 Pool
22 Solution
23 Gate electrode
24 Potentiostat
31 Linker molecule
32 Specific substance
33 Analyte
34 Electric double layer
41 Container
42 Coating layer
43 Support member
44 Porous structure
45 Nonspecific adsorption protein
46 BSA

1. A field effect transistor comprising:
a source electrode;
a drain electrode;
a channel formed between the source electrode and the drain electrode; and
a gate electrode,
wherein the channel is constituted by a single-walled carbon nanotube thin film that is grown, by a chemical vapor deposition method, using particles of a nonmetallic material as growth nuclei, the nonmetallic material containing 500 mass ppm or less metallic impurities that contain a metal and its compounds.

2. The field effect transistor according to claim 1, wherein nanodiamond is used as the growth nuclei.

3. A sensor comprising the field effect transistor according to claim 1 as a transducer.

4. The sensor according to claim 3, wherein the channel is modified with a specific substance that specifically interacts with an analyte.

5. The sensor according to claim 4, wherein the specific substance that specifically interacts with the analyte is an antibody, antibody fragment or an aptamer.

6. The sensor according to claim 4, wherein the specific substance that specifically interacts with the analyte is fixed onto the single-walled carbon nanotube thin film via linker molecules.

7. The sensor according to claim 4, wherein the analyte is cells, microorganisms, viruses, proteins, enzymes, nucleic acids or low-molecular biological substances.

8. A sensor comprising the field effect transistor according to claim 2 as a transducer.

9. The sensor according to claim 8, wherein the channel is modified with a specific substance that specifically interacts with an analyte.

10. The sensor according to claim 9, wherein the specific substance that specifically interacts with the analyte is an antibody, antibody fragment or an aptamer.

11. The sensor according to claim 5, wherein the specific substance that specifically interacts with the analyte is fixed onto the single-walled carbon nanotube thin film via linker molecules.

12. The sensor according to claim 9, wherein the specific substance that specifically interacts with the analyte is fixed onto the single-walled carbon nanotube thin film via linker molecules.

13. The sensor according to claim 10, wherein the specific substance that specifically interacts with the analyte is fixed onto the single-walled carbon nanotube thin film via linker molecules.

14. The sensor according to claim 5, wherein the analyte is cells, microorganisms, viruses, proteins, enzymes, nucleic acids or low-molecular biological substances.

15. The sensor according to claim 6, wherein the analyte is cells, microorganisms, viruses, proteins, enzymes, nucleic acids or low-molecular biological substances.

16. The sensor according to claim 9, wherein the analyte is cells, microorganisms, viruses, proteins, enzymes, nucleic acids or low-molecular biological substances.

17. The sensor according to claim 10, wherein the analyte is cells, microorganisms, viruses, proteins, enzymes, nucleic acids or low-molecular biological substances.

18. The sensor according to claim 11, wherein the analyte is cells, microorganisms, viruses, proteins, enzymes, nucleic acids or low-molecular biological substances.

19. The sensor according to claim 12, wherein the analyte is cells, microorganisms, viruses, proteins, enzymes, nucleic acids or low-molecular biological substances.

20. The sensor according to claim 13, wherein the analyte is cells, microorganisms, viruses, proteins, enzymes, nucleic acids or low-molecular biological substances.
